(19) United States
(12) Patent Application Publication
(10) Pub. No.: US 2008/0008660 A1
(43) Pub. Date: Jan. 10, 2008

(54) Antimicrobially active compounds for treating bad breath

(75) Inventors: Jurgen Rabenhorst, Hoxter (DE); Arnold Machinek, Holzminden (DE); Gerhard Schmaus, Hoxter (DE); Martina Herrmann, Hameln (DE); Gabriele Vielhaber, Holzminden (DE); Ravikumar Pillai, Emerson, NJ (US)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: Symrise GmbH & Co. KG, Holzminden (DE)

(21) Appl. No.: 11/812,085

(22) Filed: Jun. 14, 2007

Related U.S. Application Data
(63) Continuation-in-part of application No. PCT/EP06/63175, filed on Jun. 14, 2006.
(60) Provisional application No. 60/842,999, filed on Sep. 8, 2006.

(51) Int. Cl. A61K 8/30; A61K 31/16; A61Q 11/00

(52) U.S. Cl. 424/45; 424/48; 424/49; 514/617; 514/622

(57) ABSTRACT

The invention relates primarily to specific uses of a compound of the Formula 1 or of mixtures of two or more different compounds of the Formula 1, in particular for producing an antimicrobially acting agent and agent against bad breath, as well as the corresponding production methods. In addition the invention relates to specific products, in particular oral hygiene products, comprising or consisting of a compound of the Formula 1 or of a mixture of two or more different compounds of the Formula 1 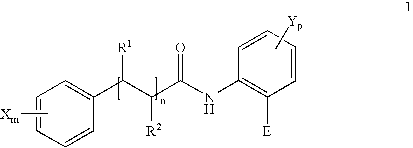 wherein the radicals of the compound of the Formula 1 or of each compound of the Formula 1 in the mixture are as defined in the preceding description.

### CROSS-REFERENCE TO RELATED APPLICATION

This application claims the benefit under 35 U.S.C. § 119(e) of provisional application Ser. No. 60/842,999, which is hereby incorporated by reference in its entirety.

### FIELD OF THE INVENTION

The present invention relates primarily to specific uses of a compound of the Formula 1 or of mixtures of two or more different compounds of the Formula 1, in particular for the production of an antimicrobially acting agent and agent against bad breath, and also corresponding methods. In addition the invention relates to specific products, in particular oral hygiene products, comprising or consisting of a compound of the Formula 1 or of a mixture of two or more different compounds of the Formula 1 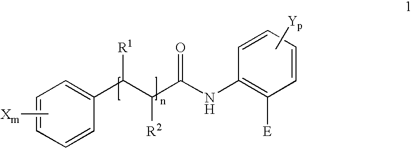
in which for the compound of the Formula 1 or for each compound of the Formula 1 in the mixture,
m=0, 1, 2 or 3,
p=0, 1 or 2,
n=0, 1 or 2, preferably n=0 or 1,
in which if n=1 or 2 then in each case the pair R¹ and R² in each case denote H or together form a further chemical bond; (such as for example in cinnamic acid derivatives),
in which if m=1, 2 or 3 each X, independently of the other, denotes OH, Oalkyl or Oacyl,
in which if p=1 or 2 each Y, independently of the other, denotes OH, Oalkyl or Oacyl,
in which E=H or denotes a radical —COOR³,
R³═H or alkyl (in particular CH₃, linear or branched alkyl chains with 2 to 30 C atoms) in which R³═H also for the corresponding pharmaceutically acceptable salts and solvates.

### BACKGROUND OF THE INVENTION

WO 2004/047833 discloses that certain anthranilic acid amides (of a Formula 1) inhibit a substance p-induced release of histamines from mast cells and are therefore suitable as cosmetic and pharmaceutical agents for the relief of itching. Some of the compounds of the Formula 1 disclosed in WO 2004/047833 are also particularly preferred for use within the scope of the present invention.

The present invention is also connected to the Applicant's Patent Application PCT/EP 2006/063175, the complete contents of which are in the course of being incorporated as a constituent part of the present application. PCT/EP 2006/063175 relates to mixtures comprising anthranilic acid amides of the Formula 1 and active cooling substances as cosmetic and pharmaceutical agents for the relief of itching.

The healthy human mucous membrane of the oral cavity and pharynx and also the solid dentine are colonised by a large number of non-pathogenic microorganisms. This so-called microflora of the oral cavity is not only harmless, but forms an important protection against opportunistic or pathogenic organisms.

A basic problem of oral hygiene is bad breath, also known as foetor ex oris or halitosis. This bad breath is formed by microorganisms by the decomposition of food residues and dead cells of the mucous membrane. The colonisation by gram-positive and gram-negative bacteria, mycobionts and/or protozoa is responsible for bad breath. In the literature anaerobic gram-negative bacteria in particular are named as the causative agent (see for example Bad Breath—A multidisciplinary Approach. Eds: D. van Steenberghe, M. Rosenberg, Leuven University Press, Leuven 1996; 111-121). Since social intercourse is often adversely affected by bad breath, there is great interest in helping those afflicted or in preventing the condition in the first place.

Gram-negative organisms belong for example to the genera Bacteroides, Fusobacterium, Haemophilus, Neisseria, Porphyromonas, Prevotella, Treponema and Veillonella.

Gram-positive bacteria are for example members of the genera Actinomyces, Eubacterium, Lactobacillus, Staphylococcus, Stomatococcus and Streptococcus.

Examples of mycobionts include for example yeasts (protoascomycetes), and moulds (plectomycetes).

Pathogenic and possibly pathogenic organisms belong for example to the group of yeasts of the Candida species (e.g. Candida albicans).

### SUMMARY OF THE INVENTION

The object of the present invention was accordingly to provide effective compounds and agents against bad breath and against the microorganisms involved in the formation of bad breath.

The invention relates primarily to the use of a compound of the Formula 1 or a mixture of two or more different compounds of the Formula 1 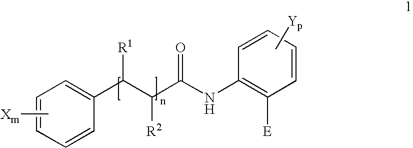
in which for the compound of the Formula 1 or for each compound of the Formula 1 in the mixture,
m=0, 1, 2 or 3,
p=0, 1 or 2,
n=0, 1 or 2,
in which if n=1 or 2 then in each case the pair R¹ and R² in each case denote H or together form a further chemical bond,
in which if m=1, 2 or 3 each X, independently of the other, denotes OH, Oalkyl or Oacyl,
in which if p=1 or 2 each Y, independently of the other, denotes OH, Oalkyl or Oacyl,
in which E=H or denotes a radical —COOR³,
R³═H or alkyl, in which R³═H also for the corresponding pharmaceutically acceptable salts and solvates,
for the production of an antibacterially acting agent.

A compound of the Formula 1 may in this connection be in the form of a suitable isomer or isomer mixture, thus for example for n=1 and R¹, R² denotes a further chemical bond, as the cis or trans isomer.

For X or Y=Oacyl, then preferably: acyl=CO—R where R═—CH₃, linear or branched alkyl radical with 2-30 C atoms.

In particular the use according to the invention relates to agents for inhibiting and/or preventing the growth and/or for destroying organisms responsible for bad breath, and/or (ii) for the treatment or prophylaxis of bad breath.

The advantageous embodiments of the compound or one of the compounds of the Formula 1 are disclosed in the sub-claims.

Accordingly compounds of the Formula 1 are preferred in which:

n=1 or 2 and the sum p+m>0

and/or p+m>0 and X or Y is selected at least once from the group comprising OH and Oacyl,

and furthermore compounds of the Formula 1 are preferred in which:

n=1,

p+m>2,

with the proviso that X and Y together are selected at least twice from the group comprising OH and Oacyl.

Also preferred are compounds of the Formula 1 in which:

n=1,

m=1, 2 or 3,

with the proviso that X is selected at least once from the group comprising OH or Oacyl

and/or

p=1 or 2,

with the proviso that Y is selected at least once from the group comprising OH and Oacyl.

Also preferred are compounds of the Formula 1 in which:

n=1

and

R¹ and R² denote in each case H or together form a further chemical bond.

For further preferred embodiments of the compounds of the Formula 1:

n=0,

also preferably:

n=0, and

m+p>2, with the proviso that at least two of the substituents X and Y are selected from the group comprising OH and Oacyl.

Similarly, compounds of the Formula 1 are preferred, in which:

n=0,

n=1,

p=0,

X═OH and

E=H.

Also preferred are compounds of the Formula 1 in which:

R³═CH₃ or linear or branched alkyl with 2 to 30 C atoms.

The present invention furthermore relates to a method for inhibiting and/or preventing the growth and/or for destroying microorganisms responsible for bad breath, comprising the following step:
contacting microorganisms responsible for bad breath with an amount, antimicrobially effective against these microorganisms, of a compound of the Formula 1 or a mixture of two or more compounds of the Formula 1 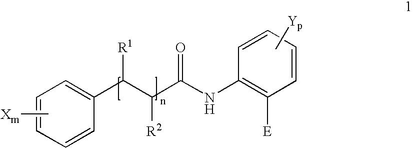
   in which for the compound of the Formula 1 or for each compound of the Formula 1 in the mixture:
   m=, 1, 2 or 3,
   p=0, 1 or 2,
   n=0, 1 or 2,
   in which if n=1 or 2 then in each case the pair R¹ and R² in each case denote H or together form a further chemical bond,
   in which if m=1, 2 or 3 each X, independently of the other, denotes OH, Oalkyl or Oacyl,
   in which if p=1 or 2 each Y, independently of the other, denotes OH, Oalkyl or Oacyl,
   in which E=H or denotes a radical —COOR³,
   R³═H or alkyl, in which R³═H also for the corresponding pharmaceutically acceptable salts and solvates.

Here too the advantageous embodiments of the compound or one of the compounds of the Formula 1 as described above may be used according to the invention.

The invention also relates to a method for controlling and/or preventing bad breath, comprising the following step:
introducing an amount, antimicrobially effective against microorganisms causing bad breath, of a compound of the Formula 1 or of a mixture comprising two or more compounds of the Formula 1 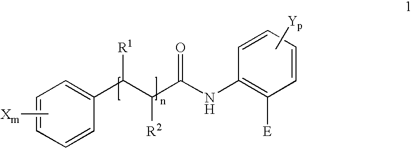
   into the oral cavity and/or the pharynx, in which for the compound of the Formula 1 or for each compound of the Formula 1 in the mixture:
   m=0, 1, 2, or 3,
   p=0, 1 or 2,
   n=0, 1 or 2,
   in which if n=0 or 2 then in each case the pair R¹ and R² in each case denote H or together form a further chemical bond;
   in which if m=1, 2 or 3 each X, independently of the other, denotes OH, Oalkyl or Oacyl,
   in which if p=1 or 2 each Y, independently of the other, denotes OH, Oalkyl or Oacyl,
   in which E=H or denotes a radical —COOR³,
   R³═H or alkyl, in which R³═H also for the corresponding pharmaceutically acceptable salts and solvates.

Here too the advantageous embodiments of the compound or one of the compounds of the Formula 1 as described above may be used according to the invention.

### BRIEF DESCRIPTION OF THE DRAWING

The FIGURE is a chart showing the results of the skin prick test/itching intensity of compositions of the invention.

### DETAILED DESCRIPTION OF THE INVENTION

The invention also relates to products that are suitable for introduction into the human oral cavity for this purpose, where they remain for a certain time and are then either swallowed, i.e. consumed (e.g. foodstuff), or can be removed from the oral cavity (e.g. chewing gum), wherein the product contains a compound of the Formula 1 or a mixture of two or more compounds of the Formula 1 in a sufficient amount to control and/or prevent bad breath. Such products also include all substances or items that are intended to be ingested in the processed, partly processed or unprocessed state by humans.

Here too the advantageous modifications of the compound or one of the compounds of the Formula 1 as described above may be employed according to the invention.

The present invention also provides oral hygiene products (oral hygiene preparations) comprising or consisting of one or a plurality of compounds of the Formula 1 to be used according to the invention, in an amount sufficient to control and/or prevent bad breath.

Here too the advantageous embodiments of the compound or one of the compounds of the Formula 1 as described above may be employed according to the invention.

Oral hygiene products are understood in the present invention to include the formulations generally known to the person skilled in the art for the cleansing and for the care of the oral cavity and pharynx as well as for freshening the breath. Known and conventional oral hygiene formulations include cremes, gels, pastes, foams, emulsions, suspensions, aerosols, sprays, as well as capsules, granules, lozenges, tablets, sweets or chewing gum, though this list of forms of administration and possibilities of use should not be regarded as limiting. Such formulations serve to clean and care for the dentine and oral cavity and also to freshen the breath.

Oral hygiene products according to the invention are preferably selected from the group consisting of: dental cremes, toothpastes, tooth gels, mouthwashes, mouth rinses, liquids for gargling, mouth sprays or throat sprays (pump-action or aerosol sprays), sucking lozenges, sucking tablets, sweets, chewing gums, chewing sweets and dental-care chewing gums.

Also preferred are oral hygiene products selected from the group consisting of dental cremes, toothpastes, tooth gels, mouth or throat sprays (pump-action or aerosol sprays), sucking lozenges, sucking tablets, sweets, chewing gums, chewing sweets and dental-care chewing gums.

The present invention furthermore provides oral hygiene products (oral hygiene preparations), comprising or consisting of a compound of the Formula 1 or a mixture of two or more compounds of the Formula 1 in an amount sufficient to control and/or prevent bad breath, with the proviso that the oral hygiene product does not comprise a mouthwash containing a mixture comprising or consisting of:
(a) one or more compounds of the Formula 1A, 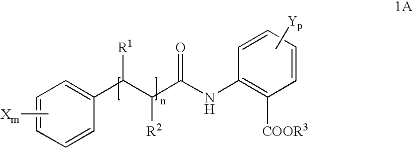
in which for the compound or each compound of the Formula 1A:
m=0, 1, 2 or 3,
p=0, 1 or 2,
n=0, 1 or 2,
in which if n=1 or 2 then in each case the pair R¹ and R² in each case denote H or together form a further chemical bond;
in which if m=1, 2 or 3 each X, independently of the other, denotes OH, Oalkyl or Oacyl,
in which if p=1 or 2 each Y, independently of the other, denotes OH, Oalkyl or Oacyl,
R³═H or alkyl, in which R³═H also for the corresponding pharmaceutically acceptable salts and solvates,
and
(b) one or more cooling substances.

A compound of the Formula 1A may in this connection be present in the form of a suitable isomer or isomer mixture, i.e. for example for n=1 and R¹, R² denotes a further chemical bond, as cis or trans isomer.

For X or Y=Oacyl, preferably acyl ═CO—R with

R═—CH₃, linear or branched alkyl radical with 2-30 C atoms.

The present invention also provides a mouthwash comprising a mixture according to the invention comprising or consisting of:
(a) one or more compounds of the Formula 1A, 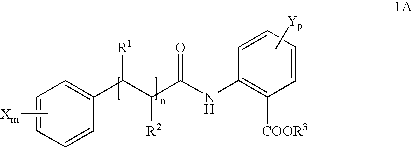
in which for the compound or each compound of the Formula 1A:
m=0, 1, 2 or 3,
p=0, 1 or 2,
n=0, 1 or 2,
in which if n=1 or 2 then in each case the pair R¹ and R² in each case denote H or together form a further chemical bond;
in which if m=1, 2 or 3 each X, independently of the other, denotes OH, Oalkyl or Oacyl,
in which if p 1 or 2 each Y, independently of the other, denotes OH, Oalkyl or Oacyl,
R³═H or alkyl, in which R³═H also for the corresponding pharmaceutically acceptable salts and solvates,
and
(b) one or more cooling substances,
in which the compound or one of the compounds of the Formula 1A is contained in a sufficient amount to control and/or prevent bad breath.

The compounds of the Formula 1A and in particular also the mixtures containing (a) one or more compounds of the Formula 1A and (b) one or more cooling substances in addition help to reduce itching and/or erythema, in which a synergistic intensification of this effect is produced by the mixture of (a) and (b), so that even very low application concentrations of compound(s) of the Formula 1A and of the further compound are sufficient to achieve a good itching-reducing effect and erythema-reducing effect.

As regards the use of the compound(s) of the Formula 1A in the mixture according to the invention, particularly preferred are compounds of the Formula 1A in which:

n=1 or 2 and the sum p+m>0

and/or

p+m>0 and X or Y is selected at least once from the group consisting of OH and Oacyl.

It is particularly preferred to use a compound of the Formula 1A in which:

n=1,

p+m≧2

with the proviso that X and Y together are chosen at least twice from the group consisting of OH and Oacyl.

Also preferred is the use of a compound of the Formula 1A in which:

n=1,

in which in addition:

m=1, 2 or 3,

with the proviso that X is chosen at least once from the group consisting of OH or Oacyl,

and/or

p=1 or 2,

with the proviso that Y is chosen at least once from the group consisting of OH and Oacyl.

If n has the value 1, then R¹ and R² in each case preferably denote H, though R¹ and R² may also together denote a further chemical bond.

The above comments basically refer to compounds of the Formula 1 where n=1.

However, the use of compounds of the Formula 1 where

n=0 is in many cases preferred.

In this case, preferably:
m+p≧2,
with the proviso that at least two of the substituents X and Y are selected from the group consisting of OH and Oacyl.

In the compounds represented by their formula and that are particularly preferred, in each case R³═H.

Instead of these preferred compounds, there may also preferably be used in each case the corresponding compounds where: R³═CH₃ or linear or branched alkyl with 2 to 30 C atoms.

Individual preferred cooling agents for use within the scope of the present invention are listed hereinafter. The person skilled in the art can amplify the following list by a large number of further cooling agents: the listed cooling agents may also be used in combination with one another: l-menthol, d-menthol, racemic menthol, menthone glycerol acetal (trade name: Frescolat®MGA), menthyl lactate (trade name: Frescolat®ML), wherein preferably menthyl lactate is l-menthyl lactate, especially l-menthyl-l-lactate), substituted menthyl-3-carboxylic acid amides (e.g. menthyl-3-carboxylic acid N-ethylamide), 2-isopropyl-N-2,3-trimethyl butanamide, substituted cyclohexanecarboxylic acid amides, 3-menthoxypropane-1,2-diol, 2-hydroxyethylmenthyl carbonate, 2-hydroxypropylmenthyl carbonate, N-acetylglycine menthyl ester, isopulegol, menthylhydroxycarboxylic acid esters (e.g. menthyl-3-hydroxybutyrate), monomenthyl succinate, 2-mercaptocyclodecanone, menthyl-2-pyrrolidin-5-one carboxylate, 2,3-dihydroxy-p-menthane, 3,3,5-trimethylcyclohexanone glycerol ketal, 3-menthyl-3,6-di- and trioxaalkanoates, 3-menthyl methoxyacetate, icilin.

On account of their particularly synergistic effect, preferred cooling agents are: l-menthol, d-menthol, racemic menthol, menthone glycerol acetal (trade name: Frescolat®MGA), menthyl lactate, preferably 1-menthyl lactate, in particular l-menthyl-l-lactate (trade name: Frescolat®ML), substituted menthyl-3-carboxylic acid amides (e.g. menthyl-3-carboxylic acid N-ethyl amide), 2-isopropyl-N-2,3-trimethyl butanamide, substituted cyclohexanecarboxylic acid amides, 3-menthoxypropane-1,2-diol, 2-hydroxyethylmenthyl carbonate, 2-hydroxypropylmenthyl carbonate, isopulegol.

Particularly preferred cooling agents are: l-menthol, racemic menthol, menthone glycerol acetal (trade name: Frescolat®MGA), menthyl lactate (preferably l-menthyl lactate, in particular 1-menthyl-l-lactate, trade name: Frescolat®ML), 3-menthoxypropane-1,2-diol, 2-hydroxyethylmenthyl carbonate, 2-hydroxypropylmenthyl carbonate.

Most particularly preferred cooling agents are: l-menthol, menthone glycerol acetal (trade name: Frescolat®MGA), menthyl lactate (preferably l-menthyl lactate, in particular 1-menthyl-l-lactate, trade name: Frescolat®ML).

The application concentration of the compounds of the Formula 1A to be used according to the invention to relieve itching may particularly—depending on the substance—be in the concentration range from 0.0001 to 10 weight percent, as is also already the case according to WO 2004/047833. However, it is preferred to use a low concentration of the compound or compounds of the Formula 1A. In particular a concentration range of 0.001 to 1 weight percent is preferred, and a range of 0.01 to 0.2 weight percent is particularly preferred, in each case referred to the total weight of a ready-for-use cosmetic or pharmaceutical end product.

The application concentration of the cooling agents to be used according to the invention to relieve itching is, depending on the substance, preferably in the concentration range from 0.01 to 20 weight percent and more preferably in the concentration range from 0.1 to 5 weight percent, referred to the total weight of a ready-for-use cosmetic or pharmaceutical end product.

Particularly preferred are mouthwashes according to the invention in which the weight ratio of the total amount of compounds of the Formula 1A to the total amount of cooling agents is in the range from 1:100 to 1:2, preferably in the range from 1:50 to 1:5 and particularly preferably in the range from 1:30 to 1:10. The proportion by weight of the cooling agents thus preferably predominates compared to the proportion by weight of the compounds of the Formula 1A.

The mouthwashes according to the invention can be combined with a large number of further constituents, whereby preferred cosmetic and/or pharmaceutical mixtures or products are obtained.

The compounds of the Formula 1 or of the Formula 1A to be used according to the invention are compounds that can be incorporated largely universally in a very wide range of application forms of oral hygiene products, without wishing to specify any particular one or a few particular application forms, i.e. the compounds of the Formula 1 or of the Formula 1A to be used according to the invention harmonise with a large number of conventional cosmetic auxiliary substances and additives. Compounds of the Formula 1 and/or 1A may if necessary be (pre)dissolved in high concentration in aprotic, dipolar solvents such as for example dimethyl sulfoxide, dimethyl formamide, but also in other solvents or solvent combinations.

It was found that when using the compounds of the Formula 1 or Formula 1A to be used according to the invention, the growth of microorganisms in the oral cavity, in particular of gram-positive and gram-negative bacteria, can be prevented or suppressed.

It has also been found that the compounds of the Formula 1 or Formula 1A to be used according to the invention can effectively reduce or eliminate the formation of bad breath, or can prevent its occurrence.

When using the compounds of the Formula 1 or Formula 1A to be used according to the invention and oral hygiene products comprising or consisting of one or more compounds of the Formula 1 or Formula 1A, an effective control of bad breath can be achieved without thereby noticeably harming the physiological flora of the oral cavity and pharynx.

The prior art did not provide any indication of the use according to the invention of compounds of the Formula 1 or Formula 1A as a means for reducing, eliminating or preventing the formation of bad breath, as well as bacterial phenomena such as for example dental caries, parodontitis, plaque and gingivitis.

Preferred compounds of the Formula 1 or Formula 1A to be used according to the invention are:

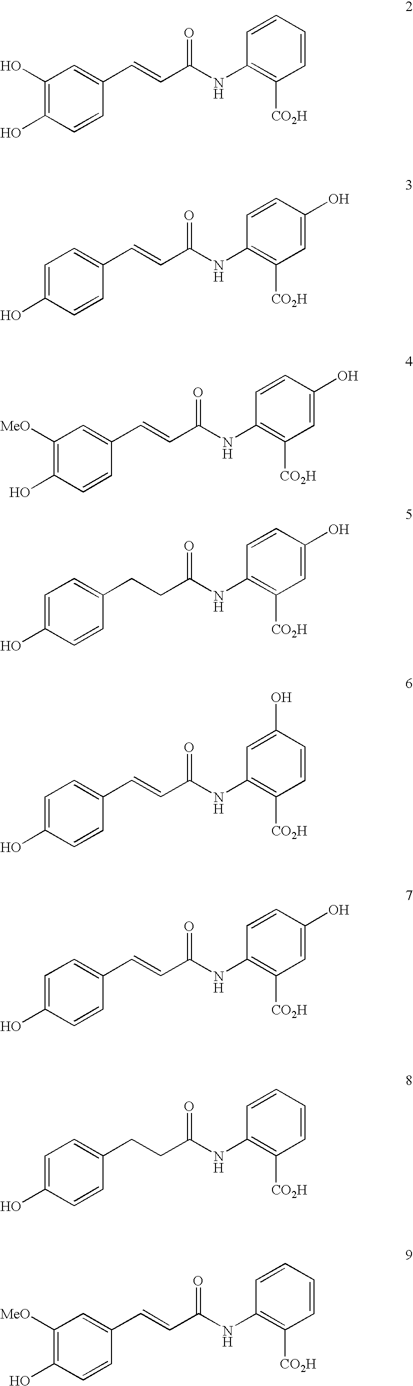

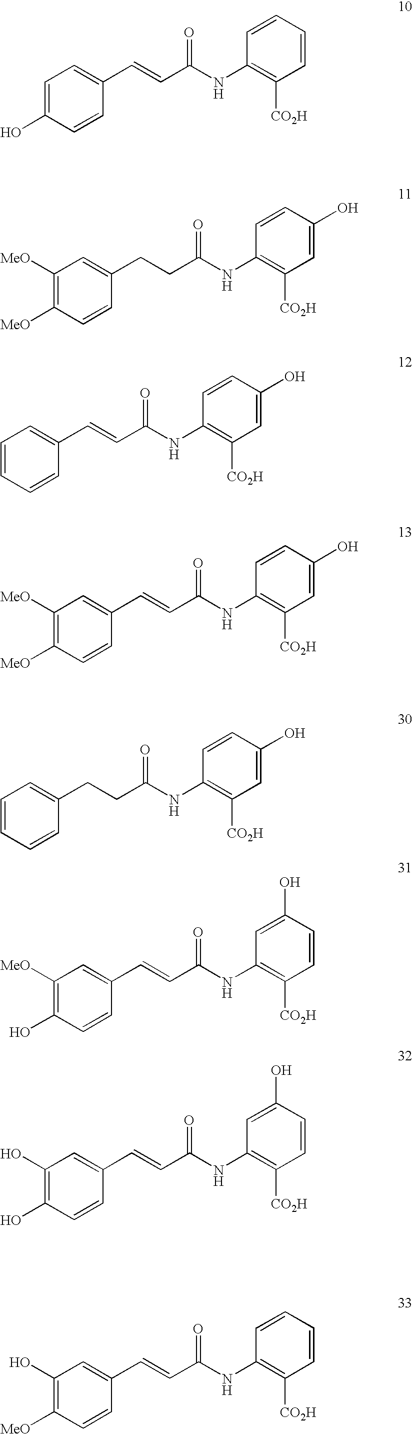

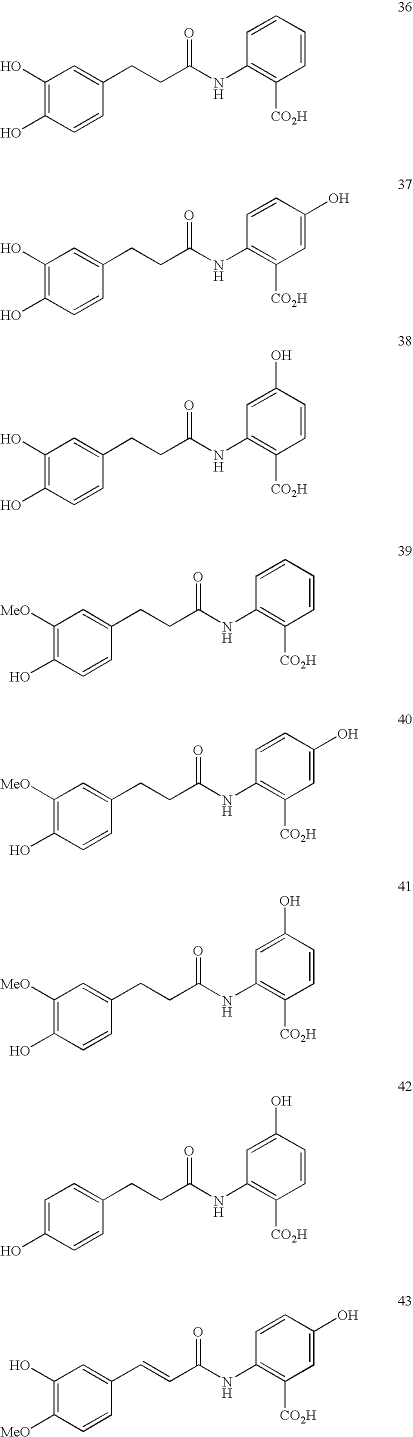

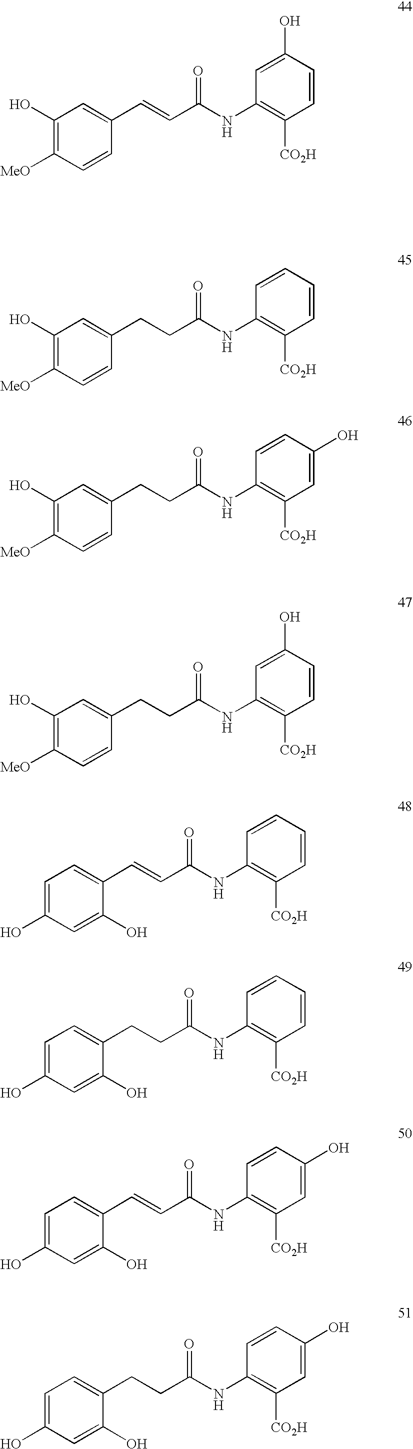

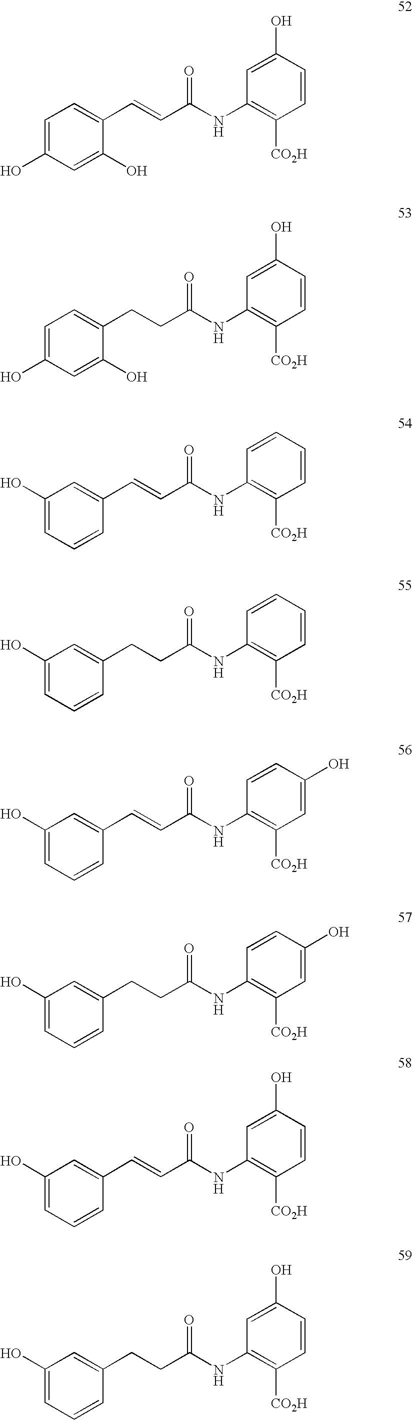

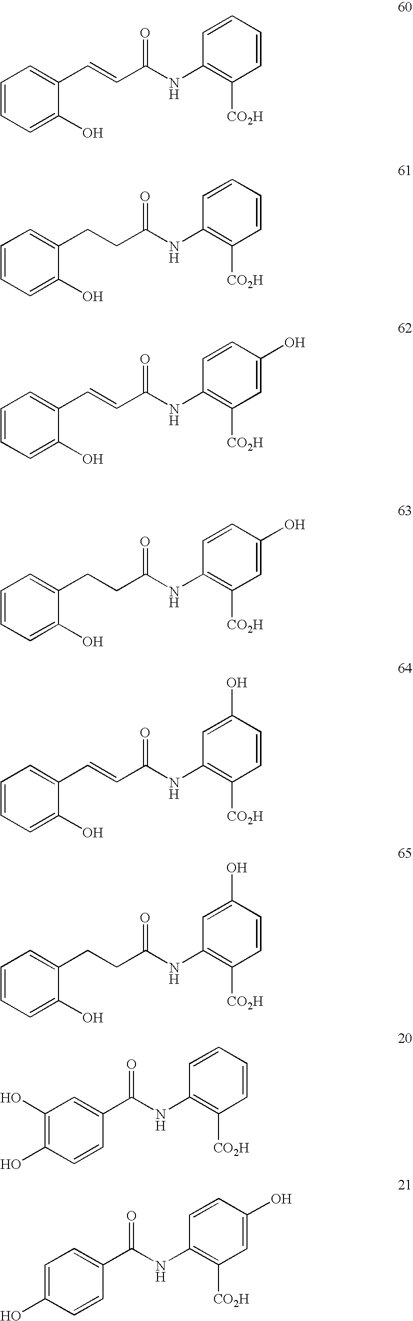

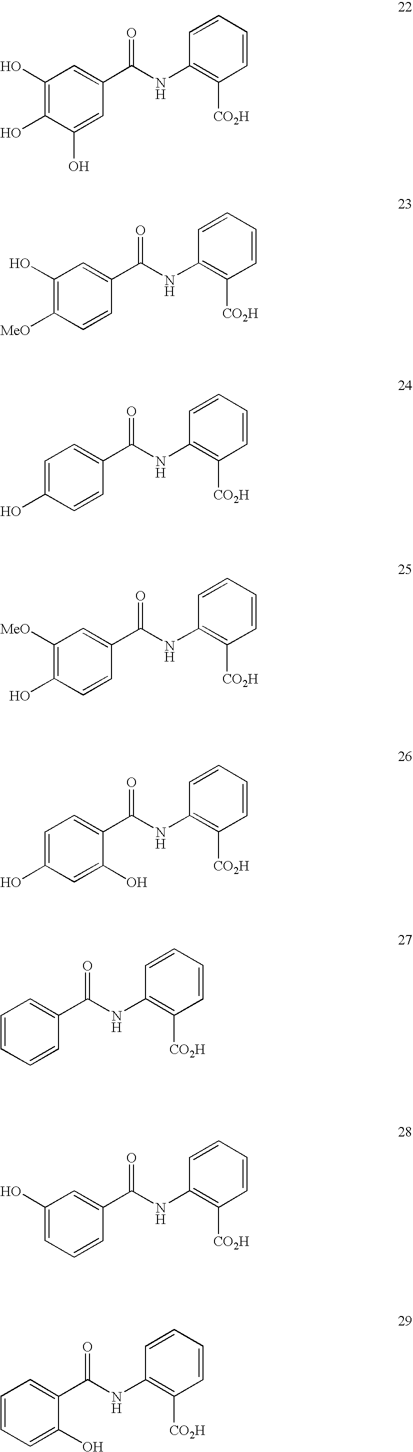

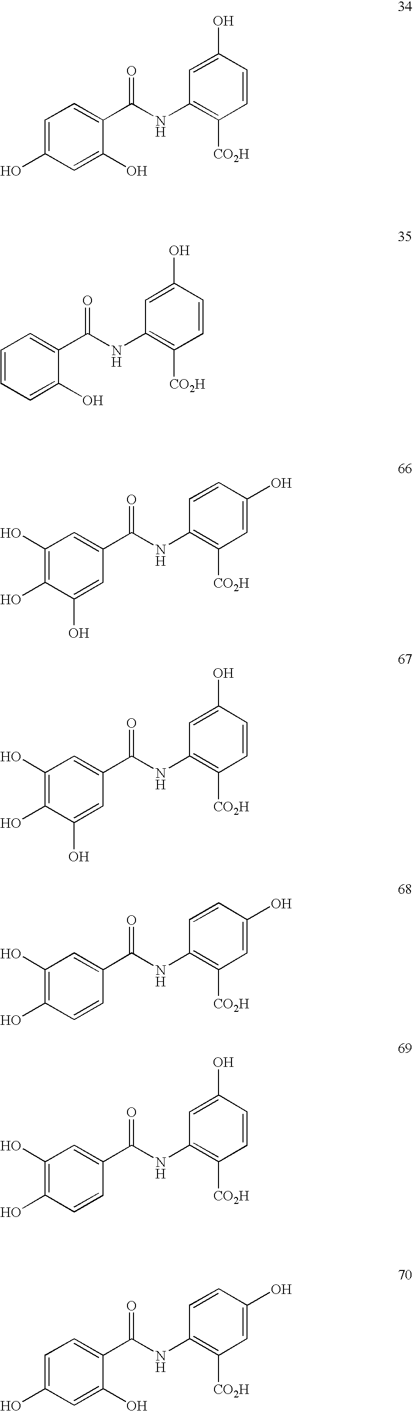

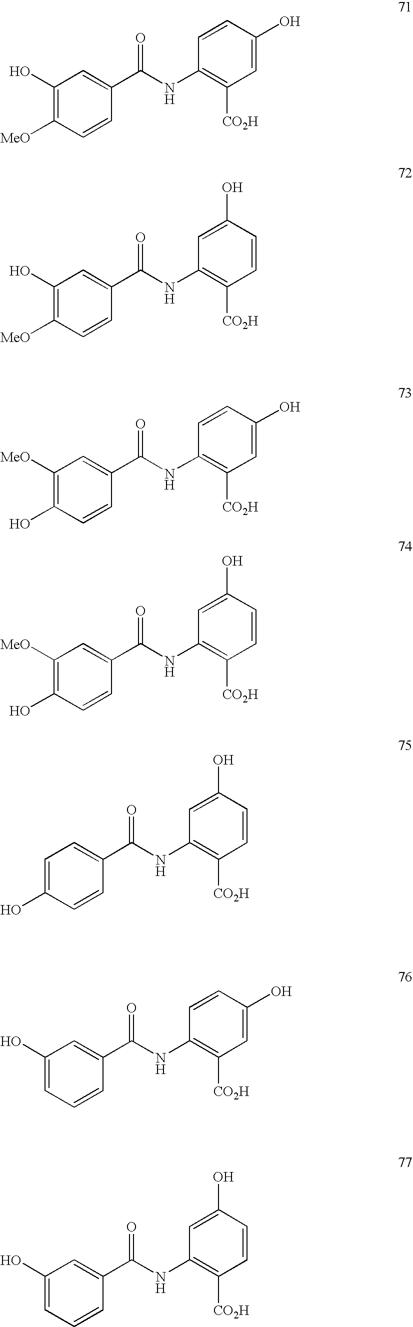

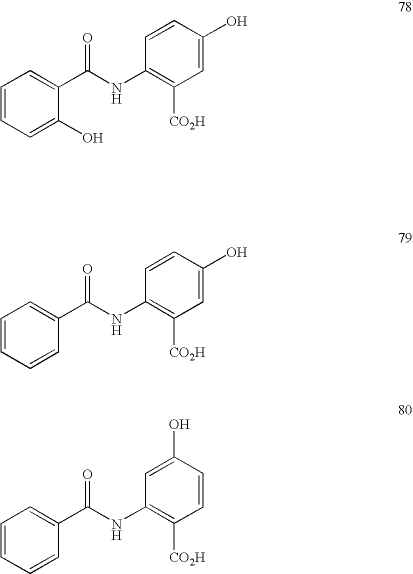

Preferred compounds of the Formula 1 to be used according to the invention are:

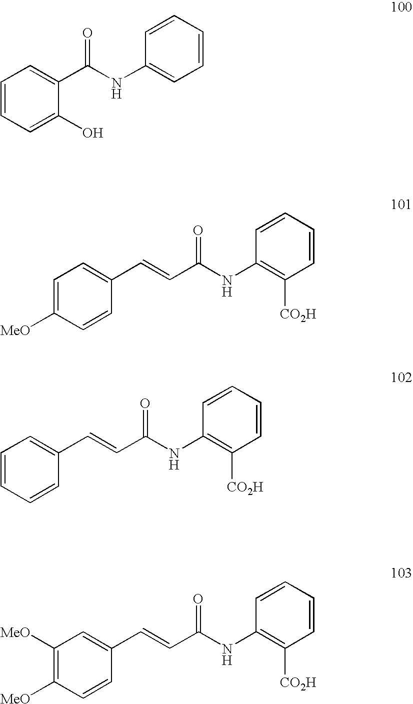

Particularly preferred compounds of the Formula 1 to be used according to the invention on account of their very effective action against anaerobic gram-negative bacteria are:

Compounds of the formulae: 28, 102, 24, 25, 10, 12, 101, 27, 29, 26, 23, 3, 100, 7, 9, 2, 8, 20, 4, 13, 75, 102 and 103.

Most preferred in this connection are in turn the compounds of the formulae 20, 8, 2, 9, 7, 100, 23, 26, 29, 27, 101, 12, 10, 25, 24, 102, 28 and 3 that are particularly effective against bad breath (see also in this connection the results of the in vitro tests on reducing bad breath:

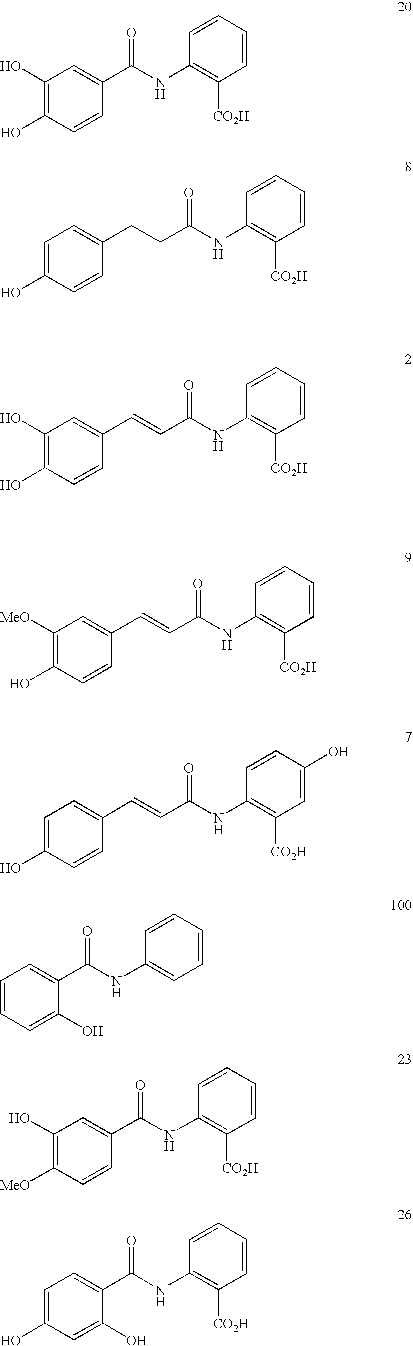

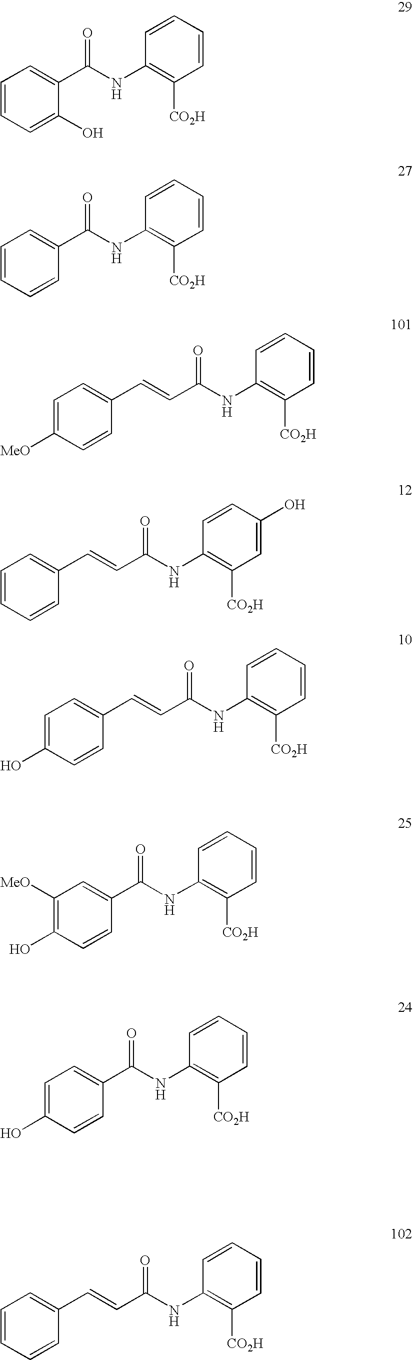

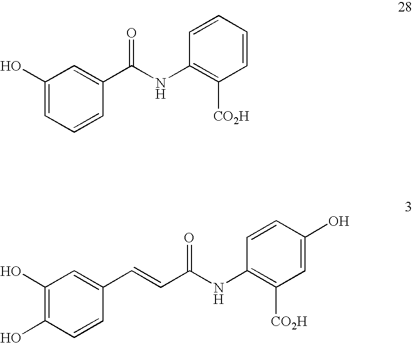

Particularly preferred compounds of the Formula 1A to be used according to the invention on account of their very effective action against anaerobic gram-negative bacteria are: 28, 24, 25, 10, 12, 27, 29, 26, 23, 3, 7, 9, 2, 8, 20, 4, 13 and 75.

Most preferred in this connection are in turn the compounds of the formulae 20, 8, 2, 9, 7, 23, 26, 29, 27, 12, 10, 25, 24, 28 and 3 that are particularly effective against bad breath (see also in this connection the results of the in vitro tests on reducing bad breath.

It is likewise advantageous to use natural or synthetic substances or substance mixtures that are characterised by an effective content of the compounds according to the invention of the Formula 1 or of the Formula 1A, such as extracts of the genera Avena, Dianthus, Silene or Melandrium. The compounds according to the invention may be used individually or in combination with other compounds according to the invention and with further aroma substances. Apart from the respective individual compounds, it is also preferred to use combinations of two or three compounds according to the invention and optionally further aroma substances.

It was found that the compounds according to the invention reduce and/or prevent the growth of gram-positive and gram-negative bacteria, mycobionts and/or protozoa of the oral cavity and larynx, preferably those bacteria, mycobionts and/or protozoa that cause bad breath.

It was also found that the compounds according to the invention prevent and/or reduce the formation of components causing bad breath.

In particular the compounds of the Formula 1 or Formula 1A used according to the invention are capable of reducing and/or preventing the growth of organisms causing bad breath, in particular of the genera Eubacterium, Fusobacterium, Haemophilus, Neisseria, Porphyromonas, Prevotella, Treponema and Veillonella, in particular Fusobacterium nucleatum, Porphyromonas endodontalis, Porphyromonas gingivalis, Prevotella intermedia, Prevotella loeschii, Treponema denticola and Veillonella parvula.

Furthermore it was surprising that the compounds of the Formula 1 or of the Formula 1A according to the invention are extremely effective against the particularly pronounced morning bad breath that is typically noticeable in the morning after getting up.

The compounds of Formula 1 or of the Formula 1A according to the invention are preferably used in oral hygiene products (oral hygiene preparations) in a total amount in the range from 0.0005-5.0 wt. % (corresponding to 5-50,000 ppm), particularly preferably in the range from 0.001-2.0 wt. % (corresponding to 10-20,000 ppm), and especially in the range from 0.0025-1.5 wt. % (corresponding to 25-15,000 ppm), in each case referred to the total weight of the preparation. In addition, the total weight of compounds of the Formula 1 or of the Formula 1A to be used according to the invention is preferably in the range from 0.005-1.0 wt. % (corresponding to 50-10,000 ppm), particularly preferably in the range from 0.01-0.5 wt. % (corresponding to 100-5,000 ppm), in each case referred to the total weight of the preparation.

In investigations carried out by the Applicants it was furthermore found that the compounds of the Formula 1 or of the Formula 1A or mixtures of two or more different compounds of the Formula 1 or of the Formula 1A to be used according to the invention have either only a slight or (substantially) neutral intrinsic taste, in particular in the concentrations specified above for oral hygiene products, whereby the compounds of the Formula 1 or of the Formula 1A can ideally be incorporated in products to be used orally such as oral hygiene products, without (noticeably) changing the intrinsic taste of the latter.

It is advantageous to buffer the oral hygiene preparations according to the invention. A pH range of 3.5-10.0 is advantageous. It is particularly expedient to choose a pH value in the range from 6.5 to 8.0.

The compounds of the Formula 1 or of the Formula 1A according to the invention can be incorporated without any problem in conventional oral hygiene formulations for oral hygiene products. Preferred oral hygiene products include tooth cremes, toothpastes, tooth gels, mouthwashes, mouth rinses, liquids for gargling, mouth or throat sprays (pump-action or aerosol sprays), sucking lozenges, sucking tablets, sweets, chewing gums, chewing sweets and dental care chewing gums.

The oral hygiene preparations according to the invention may contain auxiliary substances (additives), such as are conventionally used in such preparations, in particular one or more substances of the following group:

preservatives, abrasives (smoothing agents), further antibacterial agents, inflammation-inhibiting agents, irritation-preventing agents, irritation-inhibiting agents, further antimicrobial agents, antioxidants, astringents, antistatics, binders, (mineral) fillers, buffers, carrier materials, chelating agents (chelate formers), cleaning agents, care agents, surface-active substances, deodorising agents, emulsifiers, enzymes, fibres, film-forming agents (film-forming substances), fixatives, foam-forming agents, substances for preventing foaming, foam stabilisers, foam boosters, gelling agents, gel-forming agents, moisture-preserving agents (moisturisers), humectants, moisture-retaining substances, bleaching agents, brighteners (e.g. hydrogen pyroxide), impregnating agents, friction-reducing agents, lubricants, smell- and/or taste-modulating agents, smell- and/or taste-reducing agents, smell- and/or taste-enhancing agents, opacifiers, plasticisers, covering agents, lightening agents, silicones, (mucous membrane)/skin cooling agents (cooling substances), (mucous membrane)/skin soothing agents (mucous membrane)/skin cleansing agents, (mucous membrane)/skin care agents, (mucous membrane)/skin healing agents, mucous membrane-protecting agents, UV filters, stabilisers, suspending agents, vitamins, fatty oils, waxes, greases, phospholipids, saturated fatty acids, singly or multiply unsaturated fatty acids, alpha-hydroxy acids, polyhydroxy acids, liquifiers, colorants, colour-protecting agents, pigments, surfactants, electrolytes, silicone derivatives, polyols, organic solvents, silicic acids, calcium carbonate, calcium hydrogen phosphate, aluminium oxide, fluorides, zinc, tin, potassium, sodium and strontium salts, pyrophosphates, hydroxyapatites.

If the oral hygiene preparation is a solution or lotion, the following for example may be used as solvents: water or aqueous solutions, oils such as triglycerides of capric or capryl acid, or also alcohols, diols or polyols of low C number and also their ethers; preferably ethanol, isopropanol, propylene glycol, glycerol, ethylene glycol. In particular mixtures of the aforementioned solvents are used.

Flavouring agents and aroma substances within the scope of the present invention are sensorially active substances, which may be volatile (aroma substances) or non-volatile (flavouring agents). The volatile aroma substances may be detected by humans both orthonasally as well as retronasally. The flavouring agents interact with the taste receptors on the tongue and are responsible for the gustatory (taste) impressions sweet, sour, salty and bitter, but apart from this other, often trigeminal stimuli, such as for example sharp, burning, cooling, tingling or prickly effects are perceived.

Flavouring agents within the scope of the present invention thus include inter alia (mucous membrane) cooling agents, (mucous membrane) warming agents, sharp-tasting substances, sweeteners, sugar substitutes, organic or inorganic acidifiers (e.g. malic acid, acetic acid, citric acid, tartaric acid, phosphoric acid), bitter principles (e.g. quinine, caffeine, limonine, amarogentine, humolones, lupolones, catechols, tannins), and edible mineral salts (e.g. sodium chloride, potassium chloride, magnesium chloride and sodium phosphates).

Advantageous aroma substances that are suitable as a constituent of the preparations according to the invention are disclosed for example in S. Arctander, Perfume and Flavor Chemicals, Vol. I and II, Montclair, N.J. 1969, own publishing house or K. Bauer, D. Garbe and H. Surburg, Common Fragrance and Flavor Materials, 4th Edition, Wiley-VCH, Weinheim 2001 and may be chosen for example from the following classes of substances: aliphatic esters (saturated and unsaturated) e.g. ethyl butyrate, allyl capronate; aromatic esters e.g. benzyl acetate, methyl salicylate; cyclic alcohols e.g. menthol; aliphatic alcohols e.g. isoamyl alcohol, 3-octanol; aromatic alcohols e.g. benzyl alcohol; aliphatic aldehydes (saturated and unsaturated), e.g. acetaldehyde, isobutyraldehyde; aromatic aldehydes e.g. benzaldehyde; vanillin; ketones e.g. menthone, carvone; cyclic ethers e.g. 4-hydroxy-5-methylfuranone; aromatic ethers e.g. p-methoxybenzaldehyde, guaiacol; lactones e.g. gamma-decalactone; terpenes e.g. limonene, linalool, terpinene, terpineol, citral. Preferably a mixture of 2, 3, 4, 5, 6, 7, 8, 9, 10 or more aroma substances is used, which in turn comprises at least one, preferably 2, 3, 4, 5 or more aroma substances from the aforementioned classes of substances.

Optically active aroma substances may in this connection be used in enantiomer-pure form or as arbitrary mixtures of the two enantiomers. The same also applies to (E)/(Z) isomers and diastereomers.

It is particularly advantageous if the preparations according to the invention contain at least one aroma substance, preferably 2, 3, 4, 5, 6, 7, 8, 9, 10 or more aroma substances, selected from the following group: menthol (preferably l-menthol and/or racemic menthol), anethol, anisole, anisaldehyde, anisalcohol, (racemic) neomenthol, eucalyptol (1,8-cineol), menthone (preferably L-menthone), isomenthone (preferably D-isomenthone), isopulegol, menthyl acetate (preferably L-menthyl acetate), menthyl propionate, carvone (preferably (−)-carvone, optionally as a constituent of a spearmint oil), methyl salicylate (optionally as a constituent of a wintergreen oil), eugenol acetate, isoeugenol methyl ether, beta-homocyclocitral, eugenol, isobutyraldehyde, 3-octanol, dimethyl sulfide, hexanol, hexanal, trans-2-hexenal, cis-3-hexenol, 4-terpineol, piperitone, linalool, 8-ocimenyl acetate, isoamyl alcohol, isovaleraldehyde, alpha-pinene, beta-pinene, limonene (preferably D-limonene, optionally as a constituent of an ethereal oil), piperitone, trans-sabinene hydrate, menthofuran, humulene, germacren D, cinnamaldehyde, mintlactone, thymol, gamma-octalactone, gamma-nonalactone, gamma-decalactone, (1,3E,5Z)-undecatriene, 2-butanone, ethyl formate, 3-octyl acetate, isoamyl isovalerianate, cis- and trans-carvyl acetate, p-cymene, damascenone, damascone, cis-rose oxide, trans-rose oxide, fenchol, acetaldehyde diethyl acetal, 1-ethoxyethyl acetate, cis-4-heptenal, cis-jasmone, methyl dihydrojasmonate, 2′-hydroxypropiophenone, menthyl methyl ether, myrtenyl acetate, 2-phenylethyl alcohol, 2-phenylethyl isobutyrate, 2-phenylethyl isovalerate, geraniol, nerol, viridiflorol.

In the case of chiral compounds the (preferred) aroma substances may be present as a racemate or as an individual enantiomer or as an enantiomer-enriched mixture.

In particular a refreshing action is achieved in the oral cavity or nasopharynx if the preparations according to the invention contain at least one aroma substance, preferably 2, 3, 4, 5 or more aroma substances, from the following group: l-menthol, racemic menthol, anethol, anisaldehyde, anisalcohol, neomenthol, eucalyptol (1,8-cineol), L-menthone, D-isomenthone, isopulegol, L-menthyl acetate, (−)-carvone, methyl salicylate, trans-2-hexenal, cis-3-hexenol, 4-terpineol, linalool, 8-ocimenyl acetate, alpha-pinene, D-limonene, (+)-menthofuran, cinnamaldehyde, menthyl methyl ether.

Menthol may in this connection be used in pure form (natural or synthetic) and/or as a constituent of natural oils and/or menthol-containing fractions of natural oils, in particular in the form of ethereal oils (i.e. oils obtained by steam distillation) of certain mentha species, in particular from Mentha arvensis (cornmint) and from Mentha piperita (peppermint), these including Mentha piperita oils with regional denominations of origin from special cultivation areas, such as Willamefte, Yakima and Madras, as well as oils of the type of the aforementioned denominations.

(−)-Carvone may in this connection be used in pure form (natural or synthetic) and/or as a constituent of natural oils and/or menthol-containing fractions of natural oils, in particular in the form of ethereal (i.e. obtained by means of steam distillation) oils of certain mentha species, in particular from Mentha cardiaca or Mentha spicata.

Anethol may in this connection be used as cis- or trans-anethol or in the form of mixtures of the isomers. Anethol may be used in pure form (natural or synthetic) and/or as a constituent of natural oils and/or anethol-containing fractions of natural oils, in particular in the form of anise oil, Japanese anise oil or fennel seed oil, or anethol-containing fractions thereof.

Eucalyptol may be used in pure form (natural or synthetic) and/or as a constituent of natural oils and/or eucalyptol-containing fractions of natural oils, for example in the form of bay leaf oil, although eucalyptus oils from Eucalyptus fruticetorum and/or Eucalyptus globulus and/or eucalyptol-containing fractions thereof are preferred.

Particularly suitable substances having a cooling and/or refreshing action in the oral cavity and/or nasopharynx are: menthol, menthone, isomenthone, 1,8-cineol (eucalyptol), (−)-carvone, 4-terpineol, thymol, methyl salicylate, L-menthyl methyl ether.

Particularly suitable aroma substances and/or flavouring agents within the scope of the present invention are also ethereal oils and extracts, tinctures and balsams, such as for example anis oil, basil oil, bergamot oil, bitter almond oil, camphor liniment, citronella oil, lemon oil; Eucalyptus-citriodora oil, eucalyptus oil, fennel oil, grapefruit oil, ginger oil, camomile oil, spearmint oil, caraway seed oil, lime oil, mandarin oil, mace oil (in particular mace flower oil=macis oil, mace oil), myrrh oil, clove oil, clove bud oil, orange-flower oil, oregano oil, parsley (seed) oil, peppermint oil, rosemary oil, sage oil (muscatel sage, Dalmation or Spanish sage oil), Japanese anise oil, thyme oil, vanilla extract, juniper oil (in particular juniper berry oil), wintergreen oil, cinnamon leaf oil, cinnamon bark oil, as well as fractions thereof and constituents isolated therefrom.

Preferred cooling substances for use within the scope of the present invention for incorporation in the preparations according to the invention are listed hereinafter. The person skilled in the art can amplify the following list by a large number of further cooling substances: the cooling substances may also be used in combination with one another. The preparations according to the invention preferably contain at least one cooling substance, preferably two or more cooling substances, selected from the group consisting of:

menthone glycerol acetal (trade name: Frescolat®MGA, Symrise GmbH & Co. KG, Germany), menthyl lactate (trade name: Frescolat®ML, Symrise GmbH & Co. KG, Germany, menthyl lactate preferably being 1-menthyl lactate, in particular 1-menthyl-l-lactate), substituted menthyl-3-carboxylic acid amides (e.g. menthyl-3-carboxylic acid-N-ethylamide, also known as WS-3), 2-isopropyl-N-2,3-trimethylbutanamide (also known as WS-23), substituted cyclohexanecarboxylic acid amides, 3-menthoxypropane-1,2-diol, 2-hydroxyethylmenthyl carbonate, 2-hydroxypropylmenthyl carbonate, N-acetylglycinementhyl ester, isopulegol, menthylhydroxycarboxylic acid esters (e.g. menthyl-3-hydroxybutyrate), monomenthyl succinate, 2-mercaptocyclodecanone, menthyl-2-pyrrolidin-5-one carboxylate, 2,3-dihydroxy-p-menthane, 3,3,5-trimethylcyclohexanone glycerol ketal, 3-menthyl-3,6-di- and -trioxaalkanoates, 3-menthyl methoxy acetate, icilin.

Particularly preferred cooling substances are: menthone glycerol acetal (trade name: Frescolat®MGA), menthyl lactate (preferably 1-menthyl lactate, in particular 1-menthyl-l-lactate, trade name: Frescolat®ML), substituted menthyl-3-carboxylic acid amides (e.g. menthyl-3-carboxylic acid N-ethylamide), 2-isopropyl-N-2,3-trimethylbutanamide, 3-menthoxypropane-1,2-diol, 2-hydroxyethylmenthyl carbonate, 2-hydroxypropylmenthyl carbonate, isopulegol and monomenthyl succinate.

According to the invention, preparations according to the invention are preferred which contain l-menthol and at least one, particularly preferably at least two, cooling substances.

Preferably a preparation according to the invention contains a mixture of flavouring agents and/or aroma substances which confers a herbal, minty, cinnamon-like, clove-like, wintergreen and/or fruity character to a preparation according to the invention.

Furthermore, it is advantageous if a preparation according to the invention includes in addition one or more cooling substances, preferably from the group of cooling substances listed above. A refreshing action in the oral cavity and/or nasopharynx is achieved to a particular degree by these preferred combinations.

According to a further preferred embodiment the flavouring agents and/or aroma substances to be employed according to the invention are, before they are used in the production of the preparations according to the invention, first of all incorporated in a matrix (carrier substance) suitable for foodstuffs and luxury foods, e.g. in the form of emulsions, liposomes, e.g. based on phosphotidyl choline, microspheres, nanospheres or also in capsules, granules, or extrudates. Particularly preferably the matrix is in this connection chosen in each case so that the flavouring agents and/or aroma substances are released in a delayed manner from the matrix so as to achieve a long-lasting effect.

Preferred matrices in which the flavouring agents and/or aroma substances are incorporated before their use in the production of the preparations according to the invention include in this connection preferably one or more materials selected from the following group: carbohydrate polymers (polysaccharides) (e.g. starch, starch derivatives, cellulose or cellulose derivatives (e.g. hydroxypropylcellulose), alginates, gellan gum, agar or carragheen), natural fats, natural waxes (e.g. beeswax, carnauba wax), proteins, e.g. gelatins, complex-forming agents (e.g. cyclodextrins or cyclodextrin derivatives, preferably beta-cyclodextrin).

The loading of the matrices with flavouring agents and/or aroma substances to be used according to the invention may vary depending on the specific requirements and desired sensorial profile. Normally the loading with flavouring agents and/or aroma substances is in the range from 1 to 60 wt. %, and usually and preferably in the range from 5 to 40 wt. %, referred to the total weight of matrix (carrier substance) and flavouring agents and/or aroma substances.

It has furthermore proved to be advantageous to convert the flavouring agents and/or aroma substances into a spray-dried form before they are used in the production of the preparations according to the invention. Individual substances or mixtures of substances can be used as matrices for the flavouring agents and/or aroma substances in spray-dried form that are to be used according to the invention. Advantageous carrier substances are carbohydrates and/or carbohydrate polymers (polysaccharides). Preferred carrier substances for the flavouring agents and/or aroma substances in spray-dried form that may be mentioned are: hydrocolloids such as starches, degraded starches, chemically or physically modified starches, modified celluloses, gum arabic, gum ghatti, tragacanth, karaya, carrageenan, guar kernel flour, carob seed flour, alginates (e.g. Na alginate), pectin, inulin or xanthan gum. Preferred carrier substances are maltodextrins as well as mixtures of maltodextrins and gum arabic, in which in each case maltodextrins with dextrose equivalent values in the range 15 to 20 are in turn advantageous. The degree of degradation of the starch is measured by the characteristic number “dextrose equivalent” (DE), which can adopt the limiting values 0 for the long-chain glucose polymer and 100 for pure glucose. The encapsulation of flavouring agents and/or aroma substances by means of spray-drying is known to the person skilled in the art, and is described for example in U.S. Pat. No. 3,159,585, U.S. Pat. No. 3,971,852, U.S. Pat. No. 4,532,145 or U.S. Pat. No. 5,124,162. Spray-dried aromas are commercially available in many different types of flavours and particle sizes.

Suitable sugar substitutes which may be a constituent of the preparations according to the invention are sugar alcohols, such as for example manitol, sorbitol and sorbitol syrup, isomalt (e.g. Palatinit®), maltite and maltite syrup, lactite, xylitol, erythritol, leucrose, arabinol, arabitol, adonitol, alditol, ducitol, iditol, and also fructooligosaccharides (e.g. Raftilose®), oligofructose or polydextrose.

Typical sweeteners that may be a constituent of the preparations according to the invention are saccharin (optionally as the Na, K or Ca salt), aspartame (e.g. NutraSweet®), cyclamate (optionally as the Na or Ca salt), acesulfam-K (e.g. Sunett®), thaumatin or neohesperidine-dihydrochalcone. Furthermore, there may also be used other sweeteners such as stevioside, rebaudioside A, glycyrrhizine, “ultra sweet”, osladin, brazzein, miraculin, pentadin, phyllodulcin, dihydrochalcone, aryl ureas, trisubstituted guanidines, glycyrrhizine, superaspartame, suosan, sucralose (trichlorogalactosuccrose, TGS), alitam, monellin or Neotame®.

Preferred spicy substances and/or substances stimulating salivation in the mouth and/or substances that produce a sensation of heat and/or a tingling sensation on the skin or on the mucous membranes, and which may be a constituent of the preparations according to the invention, are for example: capsaicin, dihydrocapsaicin, gingerols, paradols, shogaols, piperin, carboxylic acid N-vanillylamides, in particular nonanoic acid N-vanillylamide, pellitorin or spilanthol, 2-nonenoic acid amides, in particular 2-nonenoic acid N-isobutylamide, 2-nonenoic acid N-4-hydroxy-3-methoxyphenylamide, alkyl ethers of 4-hydroxy-3-methoxybenzyl alcohol, in particular 4-hydroxy-3-methoxybenzyl-n-butyl ether, alkyl ethers of 4-acyloxy-3-methoxybenzyl alcohol, in particular 4-acetyloxy-3-methoxybenzyl-n-butyl ether and 4-acetyloxy-3-methoxybenzyl-n-hexyl ether, alkyl ethers of 3-hydroxy-4-methoxybenzyl alcohol, alkyl ethers of 3,4-dimethoxybenzyl alcohol, alkyl ethers of 3-ethoxy-4-hydroxybenzyl alcohol, alkyl ethers of 3,4-methylenedioxybenzyl alcohol, (4-hydroxy-3-methoxyphenyl)acetic acid amides, in particular (4-hydroxy-3-methoxyphenyl)acetic acid N-n-octylamide, vanillomandelic acid alkylamides, ferulic acid phenethylamides, nicotinaldehyde, methyl nicotinate, propyl nicotinate, 2-butoxyethyl nicotinate, benzyl nicotinate, 1-acetoxychavicol, polygodial and isodrimeninol, and also preferred are cis- and/or trans-pellitorine according to WO 2004/000787 and WO 2004/043906, alkenecarboxylic acid N-alkylamides according to WO 2005/044778, mandelic acid alkylamides according to WO 03/106404 or alkyloxyalkanoic acid amides according to WO 2006/003210.

Preferred spicy substances and/or natural extracts producing a sensation of heat and/or a tingling sensation of the skin or of the mucous membranes and that may be a constituent of the preparations according to the invention, are for example: extracts of paprika, extracts of pepper (e.g. capsicum extract); extracts of chilli pepper, extracts of ginger roots, extracts of Aframomum melgueta, extracts of Spilanthes-acmella, extracts of Kaempferia galanga or extracts of Alpinia galanga.

Preferred substances for masking one or more unpleasant taste impressions, in particular a bitter, astringent and/or metallic taste impression or aftertaste, which may be a constituent of the preparations according to the invention, are: lactisol [2O-(4-methoxyphenyl) lactic acid] (cf. U.S. Pat. No. 5,045,336), potassium 2,4-dihydroxybenzoate (cf. U.S. Pat. No. 5,643,941), ginger extracts (cf. GB 2,380,936), neohesperidine dihydrochalcone (cf. Manufacturing Chemist 2000, July issue, pp. 16-17), certain flavones (2-phenylchrom-2-en-4-one) (cf. U.S. Pat. No. 5,580,545), certain nucleotides, such as for example cytidine-5′-monophosphates (CMP) (cf. US 2002/0177576), certain sodium salts such as sodium chloride, sodium citrate, sodium acetate and sodium lactate (cf. Nature, 1997, Vol. 387, pp. 563), a lipoprotein of β-lactoglobulin and phosphatidic acid (cf. EP-A 635 218), neodiosmine [5,7-dihydroxy-2-(4-methoxy-3-hydroxyphenyl)-7-O-neohesperidosyl-chrom-2-en-4-one] (cf. U.S. Pat. No. 4,154,862), preferably hydroxyflavanones according to EP 1 258 200, preferred compounds in turn being 2-(4-hydroxyphenyl)-5,7-dihydroxychroman-4-one (naringenine), 2-(3,4-dihydroxyphenyl)-5,7-dihydroxychroman-4-one (eriodictyol), 2-(3,4-dihydroxyphenyl)-5-hydroxy-7-methoxychroman-4-one (eriodictyol-7-methyl ether), 2-(3,4-dihydroxyphenyl)-7-hydroxy-5-methoxychroman-4-one (eriodictyol-5-methyl ether), and 2-(4-hydroxy-3-methoxyphenyl)-5,7-dihydroxychroman-4-one (homoeriodictyol), their (2S)— or (2R) enantiomers or mixtures thereof, as well as their monovalent or polyvalent phenolate salts with Na⁺, K⁺, NH₄⁺Ca²⁺, Mg²⁺ or Al³⁺ as counter-cations, or γ-aminobutyric acid (4-aminobutanoic acid, as neutral form (“internal salt”), or in the carboxylate or ammonium form) according to WO 2005/096841.

Substances which have a bitter, astringent, sticky, powdery, dry, floury, rancid or metallic taste are for example: xanthine alkaloids, xanthines (caffeine, theobromine, theophylline), alkaloids (quinine, brucine, nicotine), phenolic glycosides (e.g. salicin, arbutin), flavonoid glycosides (e.g. hesperidine, naringin), chalcones and chalcone glycosides, hydrolysable tannins (gallic acid or elagic acid esters of carbohydrates, e.g. pentagalloyl glucose), non-hydrolysable tannins (optionally galloylated catechols or epicatechol and their oligomers, e.g. proanthyocyanidines or procyanidines, thearubigenin), flavones (e.g. quercetin, taxifolin, myricetin), other polyphenols (γ-oryzanol, caffeic acid or its esters), terpenoid bifters (e.g. limonoids such as limonin or nomilin from citrus fruits, lupolone and humolones from hops, iridoids, secoiridoids), absinth from wormwood, amarogentin from gentian, metallic salts (potassium chloride, sodium sulfate, magnesium sulfate), certain pharmaceutical active substances (e.g. fluoroquinolone antibiotics, paracetamol, aspirin, beta-lactam antibiotics, ambroxol, propylthiouracil [PROP], guaifenesin), certain vitamins (for example Vitamin H, vitamins from the B group such as vitamin B1, B2, B6, B12, niacin, panthothenic acid), denatonium benzoate, sucralose octaacetate, potassium chloride, magnesium salts, iron salts, aluminium salts, zinc salts, urea, unsaturated fatty acids, in particular unsaturated fatty acids in emulsions, amino acids (e.g. leucine, isoleucine, valine, tryptophan, proline, histidine, tyrosine, lysine and phenylalanine), peptides (in particular peptides with an amino acid from the group comprising leucine, isoleucine, valine, tryptophan, proline or phenylalanine at the N or C terminus).

Substances that have a bitter, astringent, sticky, powdery, dry, floury, rancid or metallic aftertaste may belong for example to the group comprising sweeteners or sugar substitutes. The following may for example be mentioned: aspartame, neotam, superaspartame, saccharine, sucralose, tagatose, monellin, steviosides, thaumatin, miraculin, glycerrhizine and its derivatives, cyclamate and the pharmaceutically acceptable salts of the aforementioned compounds.

Advantageous additives for incorporation in the preparations according to the invention are emulsifiers (e.g. lecithins, diacylglycerols, gum arabic), stabilisers (e.g. carageenan, alginate), preservatives (e.g. benzoic acid, sorbic acid), antioxidants (e.g. tocopherol, ascorbic acid), chelating agents (e.g. citric acid), plant extracts, natural or synthetic dyes or coloured pigments (e.g. carotenoids, flavonoids, anthocyans, chlorophyll and their derivatives).

Preparations according to the invention may furthermore contain antioxidants or substances that can enhance an antioxidising action, preferably naturally occurring tocopherols and their derivatives (e.g. Vitamin E acetate), Vitamin C and its salts and derivatives (e.g. ascorbyl palmitate, Mg ascorbyl phosphate, ascorbyl acetate), Vitamin A and derivatives (Vitamin A palmitate), tocotrienols, flavonoids, alpha-hydroxy acids (e.g. citric acid, lactic acid, malic acid, tartaric acid) and their Na, Ka and Ca salts, flavonoids, quercetin, phenolic benzylamines, propyl gallate, octyl gallate, dodecyl gallate, butylhydroxyanisole (BHA, E320), butylhydroxytoluene (BHT, 2,6-di-tert.-butyl-4-methylphenol, E321), lecithins, mono- and diglycerides of edible fatty acids esterified with citric acid, carotenoids, carotenes (e.g. α-carotene, β-carotene, lycopene) and their derivatives, phytic acid, lactoferrin, EDTA, EGTA), folic acid and its derivatives, ubiquinone and ubiquinol and their derivatives, ferulic acid and its derivatives, zinc and its derivatives (e.g. ZnO, ZnSO₄), selenium and its derivatives (e.g. selenium methionine), orthophosphates and Na, Ka and Ca salts of mono-phosphoric acids as well as constituents, extracts and fractions thereof isolated from plants, e.g. From tea, green tea, algae, grapeseeds, wheat germ, camomile, rosemary, oregano.

The preparations according to the invention may for example contain the following dyes, colorants or pigments: lactoflavin (riboflavin), beta-carotene, riboflavin-5′-phosphate, alpha-carotene, gamma-carotene, cantaxanthine, erythrosin, curcumin, quinoline yellow, yellow orange S, tartrazine, bixin, norbixin (annatto, orlean), capsanthin, capsorubin, lycopene, beta-apo-8′-carotinal, beta-apo-8′-carotene acid ethyl ester, xantophylls (flavoxanthin, lutein, kryptoxanthin, rubixanthin, violaxanthin, rodoxanthin), real carmine (carminic acid, cochineal), azorubin, cochineal red A (ponceau 4 R), beetroot red, betanin, anthocyans, amaranth, patent blue V, indigotin I (indigo-carmine), chlorophylls, copper compounds of chlorophylls, brilliant acid green BS (lisamine green), brilliant black BN, Carbo medicinalis vegetabilis, titanium dioxide, iron oxides and hydroxides, calcium carbonate, aluminium, silver, gold, rubin pigment BK (litholrubin BK), methyl violet B, Victoria blue R, Victoria blue B, acilan brilliant blue FFR (brilliant wool blue FFR), naphthol green B, acilan real green 10 G (acilan real green 10 G), ceres yellow GRN, Sudan blue II, ultramarine, phthalocyanine blue, phthalocyanene green, fast acid violet R. Further, naturally obtained extracts (e.g. paprika extract, black carrot extract, red charcoal extract) may also be used for colouring purposes. Good results have also been achieved with the dyes named hereinafter, the so-called Aluminium Lakes: FD & C Yellow 5 Lake, FD & C Blue 2 Lake, FD & C Blue 1 Lake, Tartrazine Lake, Quinoline Yellow Lake, FD & C Yellow 6 Lake, FD & C Red 40 Lake, Sunset Yellow Lake, Carmoisine Lake, Amaranth Lake, Ponceau 4R Lake, Erythrosyne Lake, Red 2G Lake, Allura Red Lake, Patent Blue V Lake, Indigo Carmine Lake, Brilliant Blue Lake, Brown HT Lake, Black PN Lake, Green S Lake and their mixtures.

Suitable (mineral) fillers for incorporation into the preparations according to the invention include for example calcium carbonate, titanium dioxide, silicon dioxide, talcum, aluminium oxide, dicalcium phosphate, tricalcium phosphate, magnesium hydroxide and their mixtures.

In an advantageous embodiment the preparations according to the invention contain additives that are also used in oral hygiene products or dental care agents, and in this connection preferably at least one additive selected from the following group: abrasives (smoothing or polishing agents), such as for example silicic acids, calcium carbonates, calcium phosphates, aluminium oxides and/or hydroxyapatites, and/or surface-active substances such as e.g. sodium lauryl sulfate, sodium lauryl sarcosinate and/or cocamidopropylbetaine, and/or humectants such as for example glycerol and/or sorbitol, sweeteners such as for example saccharin, taste correctives for unpleasant taste impressions, taste correctives for taste impressions that as a rule are not unpleasant, taste-modulating substances (e.g. inositol phosphate, nucleotides such as guanosine monophosphate, adenosine monophosphate or other substances such as sodium glutamate or 2-phenoxypropionic acid), carboxymethylcellulose, polyethylene glycols, carrageenan and/or Laponite®, active substances such as for example sodium fluoride, sodium monofluorophosphate, tin difluoride, quarternary ammonium fluorides, zinc citrate, zinc sulfate, tin pyrophosphate, tin dichloride, mixtures of various pyrophosphates, triclosan, cetylpyridinium chloride, aluminium lactate, potassium citrate, potassium nitrate, potassium chloride, strontium chloride, hydrogen peroxide and/or sodium bicarbonate.

A preparation according to the invention preferably contains, apart from one or more compounds of the Formula 1 or of the Formula 1A, in addition one or more substances for improving oral hygiene, such as for example substances to control or prevent plaque, tartar or dental caries, and also further substances to control or prevent bad breath. Reference may be made in this connection to U.S. Pat. No. 5,043,154. As examples there may be mentioned Zn salts, such as Zn citrate, Zn fluoride, Sn salts, such as Sn fluorides, Cu salts, fluorides, e.g. amine fluorides, alkali metal fluorides such as Na fluoride, alkaline earth metal fluorides, ammonium fluoride, phosphates, pyrophosphates, fluorophosphates such as Na monofluorophosphate, Almonofluorophosphate and Aldifluorophosphate, alpha-ionone, geraniol, thymol, isomenthyl acetate, panthenol (provitamin B5), xylitol, allantoin, niacinamide (Vitamin B3), tocopheryl acetate (Vitamin E acetate), poloxamers.

A preparation according to the invention may, in addition to one or more compounds of the Formula 1 or of the Formula 1A, also contain one or more further antimicrobial active substances for improving oral hygiene. These antimicrobial active substances may be of a hydrophilic, amphoteric or hydrophobic nature. Preferred further antimicrobial active substances are: triclosan, chlorhexidine and its salts (e.g. its acetate, gluconate or hydrochloride), peroxides, phenols and their salts, domiphen bromide (phenododecinium bromide), bromochlorophene, Zn salts, chlorophylls, Cu salts, Cu gluconate, Cu chlorophyll, sodium lauryl sulfate, quarternary monoammonium salts such as cocoalkylbenzyldimethylammonium chloride or also pyridinium salts such as cetyl pyridinium chloride. Apart from individual active substances, mixtures of active substances or natural extracts or fractions thereof containing active substances may be employed, such as are obtainable for example from neem, berberis, fennel, green tea, marigold, camomile, rosemary, thyme, propolis or turmeric.

Preparations according to the invention that are provided for use as dental care and/or oral care products are free from cariogenic substances, in particular do not contain sucrose, glucose, lactose, hydrolysed lactose, sorbose, arabinose, xylose, mannose, maltose, galactose, maltotriose and fructose.

The following examples are intended to illustrate the present invention without however restricting it. Unless otherwise stated, all parts refer to parts by weight.

### EXAMPLES

### Example 1

### In-Vitro Test for Reducing Bad Breath

This test is based on the work of Goldberg and Rosenberg (Production of Oral Malodor in an in vitro System, S. Goldberg and M. Rosenberg, pp. 143-150, in: Bad Breath—A multidisciplinary Approach, Eds: D. van Steenberghe, M. Rosenberg, Leuven University Press, 1996) and was adapted for the purposes of better reproducibility.

A sterile liquid medium that is inoculated with fresh morning saliva is incubated for a few days at 37° C. and then smelt by a test panel.

An intensive typical smell of bad breath had formed. Non-inoculated controls only have a weak to moderate smell of bad breath.

Triclosan® in a concentration of 0.05% was added as control for the tests. After the incubation the inoculated flasks had the same smell as the non-inoculated flasks.

The use of typical aroma substances for oral care applications when used in a concentration of 0.1% in the test exhibited in most cases a very unpleasant mixed smell, consisting of a mixture of bad breath and the aroma substance. In some cases the aroma substance could no longer be detected since it had obviously been decomposed by the microorganisms in the saliva.

When 2-(benzoylamino)-benzoic acid (compound 27) was used in an amount of 0.0025 wt. %, no smell similar to that obtained with 0.05 wt. % of Triclosan® could be detected. At a concentration of 0.001 wt. % a non unpleasant smell could be detected, which did not bear any resemblance to bad breath.

The minimum effective concentrations of various compounds of the Formula 1 or of the Formula 1A at which no smell could be detected are shown hereinafter:

| | |
|---|---|
| 2-[(3-hydroxybenozyl)amino]benzoic acid (Compound 28) | 0.0005% |
| | (=5 ppm) |
| 2-{[(2E)-3-phenylprop-2-enoyl]amino}benzoic acid | 0.001% |
| (Compound 102) | (=10 ppm) |
| 2-[(4-hydroxybenzoyl)amino]benzoic acid (Compound 24) | 0.001% |
| 2-[(4-hydroxy-3-methoxybenzoyl)amino]benzoic acid | 0.001% |
| (Compound 25) | |
| 2-{[(2E)-3-(4-hydroxyphenyl)prop-2- | 0.0025% |
| enoyl]amino}benzoic acid (Compound 10) | (=25 ppm) |
| 5-Hydroxy-2-{[(2E)-3-phenylprop-2- | 0.0025% |
| enoyl]amino}benzoic acid (Compound 12) | |
| 2-{[(2E)-3-(4-methoxyphenyl)prop-2- | 0.0025% |
| enoyl]amino}benzoic acid (Compound 101) | |
| 2-(benzylamino)benzoic acid (Compound 27) | 0.0025% |
| 2-[(2-hydroxybenzoyl)amino]benzoic acid (Compound 29) | 0.0025% |
| 2-[(2,4-dihydroxybenzoyl)amino]benzoic acid | 0.0025% |
| (Compound 26) | |
| 2-[(3-hydroxy-4-methoxybenzoyl)amino]benzoic acid | 0.0025% |
| (Compound 23) | |
| 5-hydroxy-2-{[(2E)-3-(3,4-dihydroxyphenyl)prop-2- | 0.0025% |
| enoyl]amino}benzoic acid (Compound 3) | |
| Salicylanilide(Compound 100) | 0.0025% |
| 5-hydroxy-2-{[(2E)-3-(4-hydroxyphenyl)prop-2- | 0.005% |
| enoyl]amino}benzoic acid (Compound 7) | (=50 ppm) |
| 2-{[(2E)-3-(4-hydroxy-3-methoxyphenyl)prop-2- | 0.005% |
| enoyl]amino}benzoic acid (Compound 9) | |
| 2-{[(2E)-3-(3,4-dihydroxyphenyl)prop-2- | 0.005% |
| enoyl]amino}benzoic acid (Compound 2) | |
| 2-{[3-(4-hydroxyphenyl)propanoyl]amino}benzoic acid | 0.005% |
| (Compound 8) | |
| 2-[(3,4-dihydroxybenzoyl)amino]benzoic acid | 0.005% |
| (Compound 20) | |
| 5-hydroxy-2-{[(2E)-3-(4-hydroxy-3-methoxyphenyl)prop-2- | 0.01% |
| enoyl]amino}benzoic acid (Compound 4) | (=100 ppm) |
| 5-Hydroxy-2-{[(2E)-3-(3,4-dimethoxyphenyl)prop-2- | 0.01% |
| enoyl]amino}benzoic acid (Compound 13) | |
| 4-hydroxy-2-[(4-hydroxybenzoyl]amino}benzoic acid | 0.01% |
| (Compound 75) | |

Other compounds such as eugenol and thymol known to be antimicrobially active and used in a concentration of 0.1% likewise suppressed the formation of bad breath in this test, but had the disadvantage of a very pronounced intrinsic smell.

### Example 2

### Determination of the Minimal Inhibiting Concentration

The minimal inhibiting concentration (MIC) was determined by way of example for 2-(benzoylamino) benzoic acid (Compound 27) in a series dilution test against various germs found in the mouth. The results are shown in the following table:

| Organism | MIC [ppm] | Type |
|---|---|---|
| Fusobacterium nucleatum | 500 | bacteriostatic |
| Fusobacterium nucleatum | 1000 | bactericide |
| Prevotella intermedia | 500 | bacteriostatic |
| Prevotella intermedia | 1000 | bactericide |
| Staphylococcus aureus | 500 | bactericide |
| Veillonella parvula | 250 | bactericide |

A bactericidal action against the germs fusobacterium nucleatum and Prevotella intermedia causing bad breath was also found.

For example, the Compound 10 to be used according to the invention (avenanthramide D) did not have any effect at a concentration of 1,000 ppm against the germs Candida albicans, Aspergillus niger or also Escherichia coli, which are not connected with bad breath.

### Formulation Examples

F1: Gel dental creme active against bad breath

| | I (%) | II (%) | III (%) |
|---|---|---|---|
| Na carboxymethylcellulose | 0.40 | 0.40 | 0.40 |
| Sorbitol 70%, in water | 72.00 | 72.00 | 72.00 |
| Polyethylene glycol (PEG) 1500 | 3.00 | 3.00 | 3.00 |
| Na saccharinate | 0.07 | 0.07 | 0.07 |
| Na fluoride | 0.24 | 0.24 | 0.24 |
| p-hydroxybenzoic acid | 0.15 | 0.15 | 0.15 |
| (PHB)-ethyl ester | | | |
| Aroma | 1.0 | 1.00 | 1.00 |
| 2-[(3-hydroxybenozyl)amino]benzoic | 0.025 | 0.06 | 0.10 |
| acid (Compound 28) | | | |
| Abrasive silicic acid | 11.00 | 11.00 | 11.00 |
| Thickening silicic acid | 6.00 | 6.00 | 6.00 |
| Sodium dodecyl sulfate (SDS) | 1.40 | 1.40 | 1.40 |
| Water dist. | Ad 100.00 | Ad 100.00 | Ad 100.00 |

F2: Dental creme against plaque formation
and effective against bad breath

| | I (%) | II (%) | III (%) |
|---|---|---|---|
| Na carboxymethylcellulose | 1.00 | 1.00 | 1.00 |
| Glycerol | 12.50 | 12.50 | 12.50 |
| Sorbitol 70%, in water | 29.00 | 29.00 | 29.00 |
| Na saccharinate | 0.20 | 0.20 | 0.20 |
| Na fluoride | 0.22 | 0.22 | 0.22 |
| Azacycloheptane-2,2-diphospho acid, | 1.00 | 1.00 | 1.00 |
| di-sodium salt | | | |
| Bromochlorophene | 0.10 | 0.10 | 0.10 |
| Peppermint aroma | 1.10 | 1.10 | 1.10 |
| Salicylanilide (Compound 100) | 0.025 | 0.08 | 0.15 |
| Abrasive silicic acid | 15.00 | 15.00 | 15.00 |
| Thickening silicic acid | 5.00 | 5.00 | 5.00 |
| Sodium dodecyl sulfate (SDS) | 1.50 | 1.50 | 1.50 |
| Water dist. | Ad 100.00 | Ad 100.00 | Ad 100.00 |

F3: Dental creme against plaque formation
and effective against bad breath

| | I (%) | II (%) | III (%) |
|---|---|---|---|
| Carragenan | 0.90 | 0.90 | 0.90 |
| Glycerol | 15.00 | 15.00 | 15.00 |
| Sorbitol 70%, in water | 25.00 | 25.00 | 25.00 |
| PEG 1000 | 3.00 | 3.00 | 3.00 |
| Na fluoride | 0.24 | 0.24 | 0.24 |
| Tetra potassium-diphosphate | 4.50 | 4.50 | 4.50 |
| Tetrasodium-diphosphate | 1.50 | 1.50 | 1.50 |
| Na saccharinate | 0.40 | 0.40 | 0.40 |
| Precipitated silicic acid | 20.00 | 20.00 | 20.00 |
| Titanium dioxide | 1.00 | 1.00 | 1.00 |
| PHB methyl ester | 0.10 | 0.10 | 0.10 |
| Spearmint aroma | 1.10 | 1.10 | 1.10 |
| 2-(benzylamino)benzoic acid | 0.025 | 0.06 | 0.10 |
| (Compound 27) | | | |
| Sodium dodecyl sulfate | 1.30 | 1.30 | 1.30 |
| Water dist. | Ad 100.00 | Ad 100.00 | Ad 100.00 |

F4: Dental creme for sensitive teeth and active against bad breath

| | I (%) | II (%) | III (%) |
|---|---|---|---|
| Na carboxymethylcellulose | 0.70 | 0.70 | 0.70 |
| Xanthan Gum | 0.50 | 0.50 | 0.50 |
| Glycerol | 15.00 | 15.00 | 15.00 |
| Sorbitol 70%, in water | 12.00 | 12.00 | 12.00 |
| K nitrate | 5.00 | 5.00 | 5.00 |
| Na monofluorophosphate | 0.80 | 0.80 | 0.80 |
| PHB methyl ester | 0.15 | 0.15 | 0.15 |
| PHB propyl ester | 0.05 | 0.05 | 0.05 |
| Na-saccharinat | 0.20 | 0.20 | 0.20 |
| Aroma | 1.00 | 1.00 | 1.00 |
| 2-(benzylamino)benzoic acid | 0.025 | 0.06 | 0.10 |
| (Compound 27) | | | |
| Ca carbonate | 35.00 | 35.00 | 35.00 |
| Silicon dioxide | 1.00 | 1.00 | 1.00 |
| Sodium dodecyl sulfate | 1.50 | 1.50 | 1.50 |
| (SDS) | | | |
| Water dist. | Ad 100.00 | Ad 100.00 | Ad 100.00 |

F5: Dental creme for sensitive teeth and active against bad breath

| | I (%) | II (%) | III (%) |
|---|---|---|---|
| Hydroxyethylcellulose | 1.40 | 1.40 | 1.40 |
| Guar Gum | 0.60 | 0.60 | 0.60 |
| Glycerol | 18.00 | 18.00 | 18.00 |
| Sorbitol 70%, in water | 12.00 | 12.00 | 12.00 |
| Na saccharinate | 0.35 | 0.35 | 0.35 |
| Colorant | 0.01 | 0.01 | 0.01 |
| PHB methyl ester | 0.15 | 0.15 | 0.15 |
| PHB propyl ester | 0.04 | 0.04 | 0.04 |
| Sr chloride | 10.50 | 10.50 | 10.50 |
| Cinnamon aroma | 1.20 | 1.20 | 1.20 |
| 2-[(3-hydroxy-4- | 0.025 | 0.10 | 0.18 |
| methoxybenzoyl)amino]benzoic | | | |
| acid (Compound 23) | | | |
| Precipitated silicic acid | 15.00 | 15.00 | 15.00 |
| Silicon dioxide | 1.60 | 1.60 | 1.60 |
| Sodium dodecyl sulfate | 1.30 | 1.30 | 1.30 |
| Water dist. | Ad 100.00 | Ad 100.00 | Ad 100.00 |

F6: Ready-for-use mouthwash containing fluoride
and effective against bad breath

| | I (%) | II (%) | III (%) |
|---|---|---|---|
| Ethanol | 7.00 | 7.00 | 7.00 |
| Glycerol | 12.00 | 12.00 | 12.00 |
| Na fluoride | 0.05 | 0.05 | 0.05 |
| Pluronic F-127 ® (BASF, surface- | 1.40 | 1.40 | 1.40 |
| active substance) | | | |
| Na phosphate buffer pH 7.0 | 1.10 | 1.10 | 1.10 |
| Sorbic acid | 0.20 | 0.20 | 0.20 |
| Na saccharinate | 0.10 | 0.10 | 0.10 |
| Peppermint aroma | 0.15 | 0.15 | 0.15 |
| 2-(benzylamino)benzoic acid | 0.01 | 0.02 | 0.03 |
| (Compound 27) | | | |
| Colorant | 0.01 | 0.01 | 0.01 |
| Water dist. | Ad 100.00 | Ad 100.00 | Ad 100.00 |

F7: Mouthwash concentrate effective against bad breath

| | I (%) | II (%) | III (%) |
|---|---|---|---|
| Ethanol, 95% | 80.00 | 80.00 | 80.00 |
| Na cyclamate | 0.15 | 0.15 | 0.15 |
| Cinnamon aroma | 3.50 | 3.50 | 3.50 |
| Colorant | 0.01 | 0.01 | 0.01 |
| 2-{[(2E)-3-phenylprop-2- | 0.50 | 1.0 | 3.0 |
| enoyl]amino}benzoic acid | | | |
| (Compound 102) | | | |
| Water dist. | Ad 100.00 | Ad 100.00 | Ad 100.00 |

F8: Chewing gum effective against bad breath

| | I (%) | II (%) | III (%) |
|---|---|---|---|
| Chewing gum base | 21.00 | 21.00 | 21.00 |
| Glucose syrup | 16.50 | 16.50 | 16.50 |
| Glycerol | 0.50 | 0.50 | 0.50 |
| Granulated sugar | 60.45 | 60.40 | 60.30 |
| Spearmint aroma | 1.50 | 1.50 | 1.50 |
| 2-(benzylamino)benzoic acid | 0.05 | 0.15 | 0.35 |
| (Compound 27) | | | |

F9: Sugar-free chewing gum effective against bad breath

| | I (%) | II (%) | III (%) |
|---|---|---|---|
| Chewing gum base | 30.00 | 30.00 | 30.00 |
| Powdered sorbitol | 38.45 | 38.40 | 38.30 |
| Palatinite | 9.50 | 9.50 | 9.50 |
| Xylitol | 2.00 | 2.00 | 2.00 |
| Mannitol | 3.00 | 3.00 | 3.00 |
| Aspartame | 0.10 | 0.10 | 0.10 |
| Acesulfam K | 0.10 | 0.10 | 0.10 |
| Emulgum/Emulgator | 0.30 | 0.30 | 0.30 |
| Sorbitol 70%, in water | 14.00 | 14.00 | 14.00 |
| Glycerol | 1.00 | 1.00 | 1.00 |
| Cinnamon-menthol-aroma | 1.50 | 1.50 | 1.50 |
| 2-(benzylamino)benzoic acid | 0.05 | 0.10 | 0.20 |
| (Compound 27) | | | |

F10: Gelatin capsules effective against
bad breath, for direct consumption

| | I (%) | II (%) | III (%) |
|---|---|---|---|
| Gelatine casing: | | | |
| Glycerol | 2.014 | 2.014 | 2.014 |
| Gelatine 240 Bloom | 7.91 | 7.91 | 7.91 |
| Sucralose | 0.065 | 0.065 | 0.065 |
| Allura Red | 0.006 | 0.006 | 0.006 |
| Brilliant blue | 0.005 | 0.005 | 0.005 |
| Core composition: | | | |
| Vegetable oil triglyceride | 82.0 | 74.0 | 60.0 |
| Aroma B | 7.75 | 15.0 | 25.0 |
| 2-(benzylamino)benzoic acid | 0.25 | 1.0 | 5.0 |
| (Compound 27) | | | |

Aroma B has the following composition (figures in each case in wt. %):

0.1% Neotam powder, 0.05% aspartame, 29.3% peppermint oil arvensis, 29.3% peppermint piperita oil Willamette, 2.97% sucralose, 2.28% triacetin, 5.4% diethyl tartrate, 12.1% peppermint oil yakima, 0.7% ethanol, 3.36% 2-hydroxy-ethylmenthyl carbonate, 3.0% 2-hydroxypropylmenthyl carbonate, 0.27% vanillin, 5.5% D-limonene, 5.67% L-menthyl acetate.

The gelatin capsules suitable for direct consumption had a diameter of 5 mm, the weight ratio of the core material to the gelatin material being 90:10. The capsule opened in the mouth in less than 10 seconds and dissolved completely in less than 50 seconds.

The synergistic enhancement of the itching relief effectiveness of the active substance combinations according to the invention containing (a) compound(s) of the Formula 1A and (b) one or more cooling substances can be seen from the human in vivo studies (skin prick test) described hereinafter.

### Example 3

Human skin prick test for detecting the synergistically enhanced effectiveness of combinations consisting of an active substance relieving itching (dihydroavenanthramide D; CARN: 697235-49-7; benzoic acid, 2-[[3-(4-hydroxyphenyl)-1-oxopropyl]amino]-(9Cl)) and a cooling substance menthyl lactate (Trade name: Frescolat®ML)

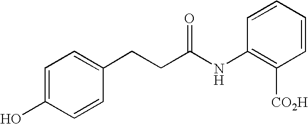

Structural formula: dihydroavenanthramide D (=Compound 8)

Description of the Test Method:

The investigations were carried out on 10 test subjects (5 test areas on the inside of the lower arm; 1× untreated, 4 application surfaces for tests)

Tests:

1. Placebo formulation

2. Product A: as placebo formulation, containing in addition 1 wt. % menthyl lactate;

3. Product B: as placebo formulation, containing in addition 0.05 wt. % dihydroavenanthramide D;

4. Product C: as placebo formulation, containing in addition 0.025 wt. % dihydroavenanthramide D and 0.5 wt. % Frescolat ML

Experimental Procedure:

A specified amount of a histamine chloride solution (HAL Allergie GmbH, Düsseldorf; concentration: 10 mg/ml) was applied to the skin on the inside of the lower arm. The skin was then gently scratched on the surface with a special lance (Feather Safety Razor; LTD Medical Division, Japan). Itching was experienced within 5 minutes. The placebo formulation as well as the products A-C were then applied (amount: 2 mg/cm²). The influence of the products A-C on relieving the itching compared to the untreated area and to the placebo was determined after 90 minutes. The experimental procedure was carried out under standardised conditions (20° C.+/−1° C.; atmospheric humidity: 50%+/−5%).

Results:

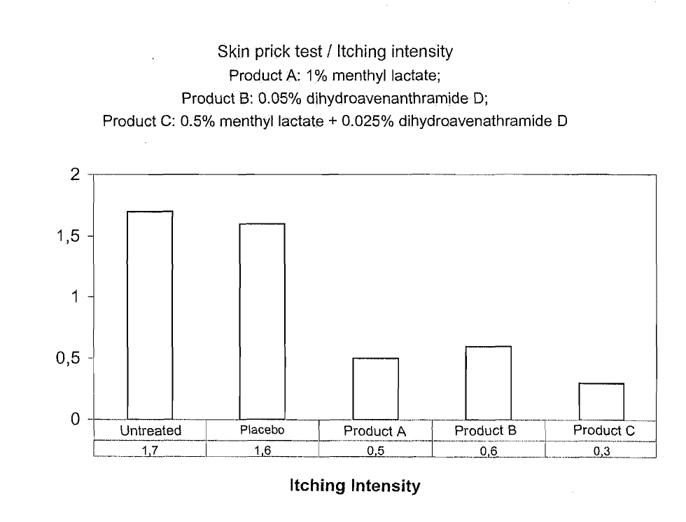

1. The products A-C led to a significantly better reduction of the itching compared to the untreated area and compared to the placebo formulation without active substance (FIG. 1)

2. Product C as opposed to the products A and B produced the best reduction in itching (FIG. 1).

The human in vivo skin prick test study shows in an exemplary manner that a better reduction in itching is achieved by the combination of anthranilic acid amides of the general Formula 1A combined with cooling substances. For product C a significantly better reduction in itching was observed compared to the products A and B (FIG. 1). The reduction in itching exceeded the values expected on a purely additive basis, thereby unambiguously demonstrating a synergistic effect of the product C containing 0.5 wt. % Frescolat ML and 0.025 wt. % dihydroavenanthramide D (Compound 8). A synergistic enhancement of the reduction in erythema achieved by product C as opposed to the products A and B was also demonstrated within the scope of the experiment.

The synergistic enhancement of the effectiveness of the active substance combination according to the invention, consisting of a cooling substance and an itching relief active substance, can be detected on the basis of the existing sensorial data by means of Kull's equation (F. C. Kull et al.; Applied Microbiology Vol. 9, p. 538-541 (1961); David C. Steinberg; Cosmetics & Toiletries Vol. 115 (No. 11), p. 59-62; November 2000; for the calculation procedure see also Table 10). Kull's equation enables the pure substances and the active substance mixtures prepared therefrom to be compared as regards their itching relief effectiveness. The so-called synergy index (SI) is calculated thereby, which is a measure of a synergistic, but also of any possible antagonistic effect, of an itching relief mixture. A synergistic effect is evident if the calculated Si value is less than 1. If an SI value of exactly 1 is found, this indicates on the one hand a purely additive effect of two itching relief products. If the SI value is larger than 1, this indicates on the other hand an (often undesirable) antagonistic effect.

The calculation of the SI value for the treatment with a mixture consisting of menthyl lactate and dihydroavenanthramide D after an incubation phase of 90 minutes is shown hereinafter by way of example. The calculated Si value of 0.066 clearly shows that the mixture consisting of menthyl lactate and dihydroavenanthramide D forms a highly synergistic active substance combination.

TABLE 1
Calculation of the synergy index (SI) of a menthyl lactate/dihydroavenanthramide
D mixture (product C) consisting of the comparison cooling substance (A) and
the comparison itching relief active substance dihydroavenanthramide D (product B)

| | | | C |
|---|---|---|---|
| | | | Menthyl lactate |
| | A | B | (0.5 wt. %) + |
| | Menthyl lactate | Dihydroaven- | dihydroaven- |
| | (Frescolat ML) | anthramide D | anthramide D |
| | 1 wt. -% | 0.05 wt. % | (0.025 wt. %) |
| Itching relief | 0.5 | 0.6 | 0.3 |
| (intensity scale: 0-2) | | | |
| 2 = severe itching | | | |
| 0 = slight itching | | | |
| Kull's equation: SI = C × D/A + C × E/B | | | |
| Itching relief | 0.5 | | |
| Product A | | | |
| Itching relief | 0.6 | | |
| Product B | | | |
| Itching relief | 0.3 | | |
| Product C | | | |
| D: Proportion of | 0.5 | | |
| A in C | | | |
| E: Proportion of | 0.5 | | |
| B in C | | | |
| SI: Synergy Index | 0.55 | | |

Literature:
synergy index:
D. C. Steinberg; Cosmetics & Toiletries 115 (11); p. 59-62 (2000)
F. C. Kull et al.; Applied Microbiology 9; p. 538-541 (1961)

1. An antimicrobial agent comprising a compound of the Formula 1 or of a mixture of two or more different compounds of the Formula 1 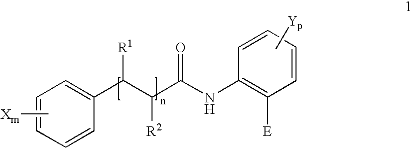
in which for the compound of the Formula 1 or for each compound of the Formula 1 in the mixture,
m=0, 1, 2 or 3,
p=0, 1 or 2,
n=0, 1 or 2,
in which if n=1 or 2 then in each case the pair R¹ and R² in each case denote H or together form a further chemical bond,
in which if m=1, 2 or 3 each X, independently of the other, denotes OH, Oalkyl or Oacyl,
in which if p=1 or 2 each Y, independently of the other, denotes OH, Oalkyl or Oacyl,
in which E=H or denotes a radical —COOR³,
R³═H or alkyl, in which R³═H also for the corresponding pharmaceutically acceptable salts and solvates.

2. The antimicrobial agent according to claim 1, in which the agent is an agent (i) for inhibiting and/or preventing the growth and/or for destroying organisms causing bad breath, and/or (ii) for controlling or preventing bad breath.

3. The antimicrobial agent according to claim 2, in which the microorganisms causing bad breath are selected from the group consisting of: eubacterium, fusobacterium, haemophilus, neisseria, porphyromonas, prevotella, treponema and veillonella species.

4. The antimicrobial agent according to claim 1, in which for the compound or one of the compounds of the Formula 1:
n=1 or 2 and the sum p+m>0
and/or p+m>0 and X or Y is selected at least once from the group consisting of OH and OAcyl.

5. The antimicrobial agent according to claim 1, in which for the compound or one of the compounds of the Formula 1:
n=1,
p+m≧2,
with the proviso that X and Y are chosen at least twice from the group consisting of OH and OAcyl.

6. The antimicrobial agent according to claim 1, in which for the compound or one of the compounds of the Formula 1:
n=1,
m=1, 2 or 3,
with the proviso that X is chosen at least once from the group consisting of OH or OAcyl
and/or
p=1 or 2,
with the proviso that Y is chosen at least once from the group consisting of OH and OAcyl.

7. The antimicrobial agent according to claim 1, in which for the compound or one of the compounds of the Formula 1:
n=
and
R¹ and R² in each case denote H or together form a further chemical bond.

8. The antimicrobial agent according to claim 1, in which the compound or one of the compounds of the Formula 1 is selected from the group consisting of:

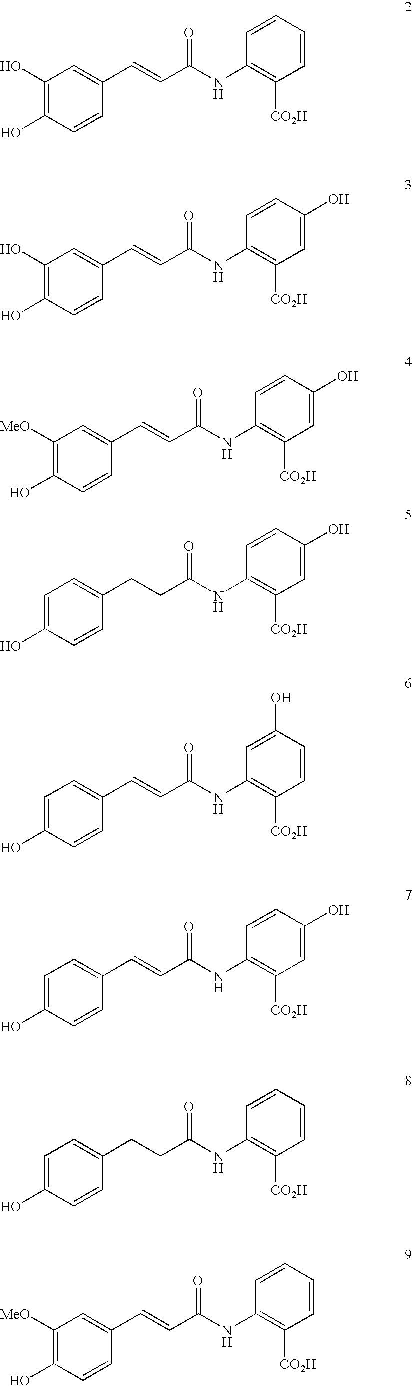

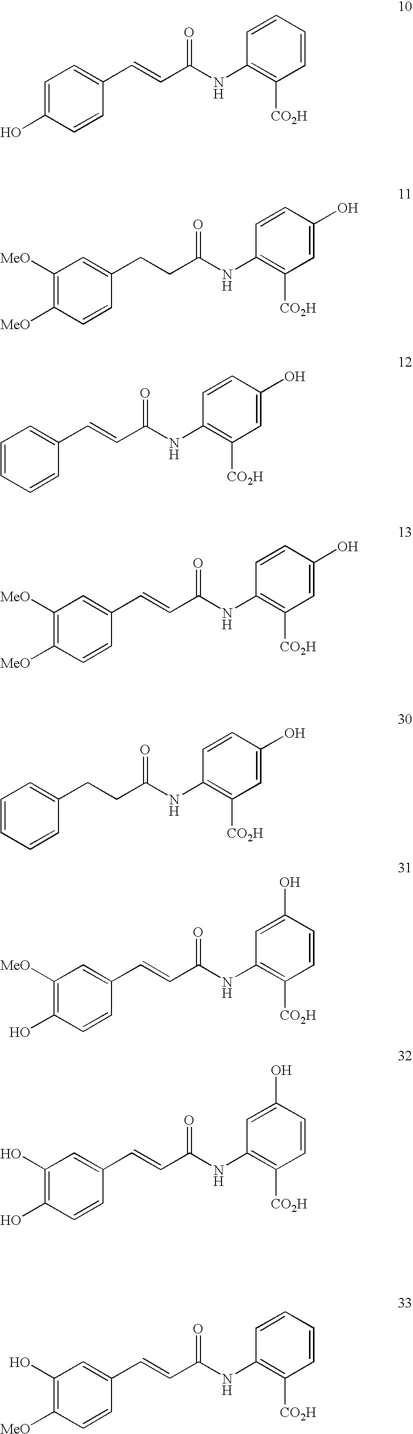

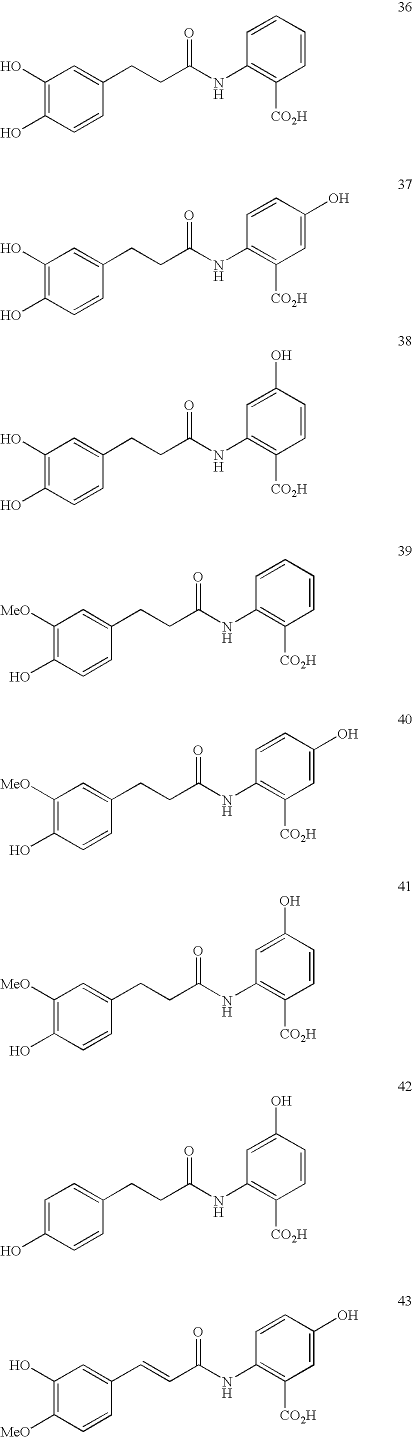

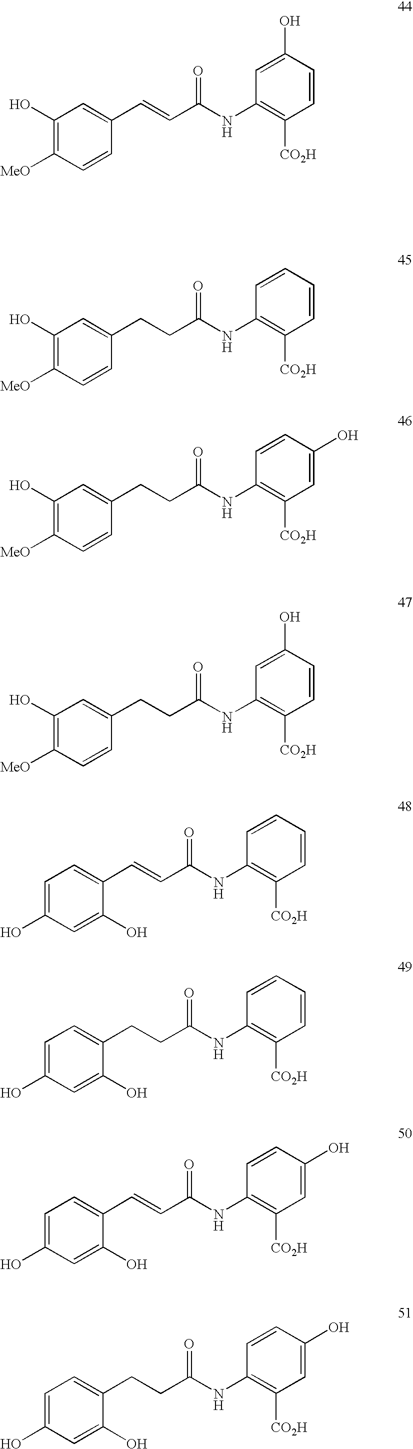

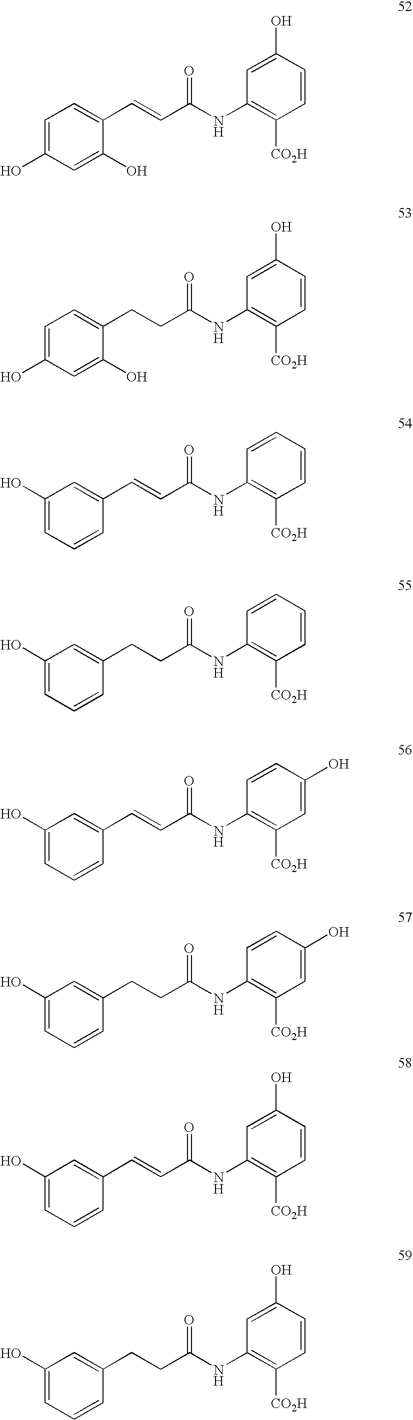

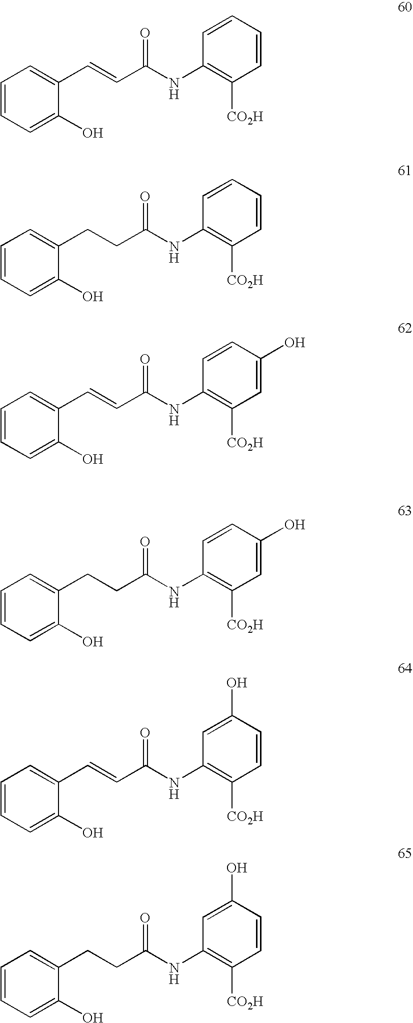

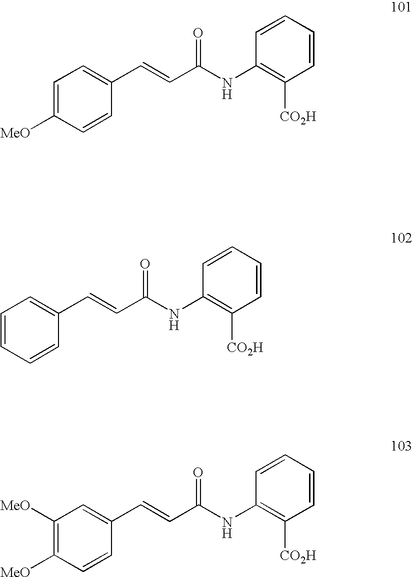

9. The antimicrobial agent according to claim 1, in which for the compounds of Formula 1:
n=0.

10. The antimicrobial agent according to claim 9, in which for the compounds of Formula 1:
m+p>2,
with the proviso that at least two of the substituents X and Y are selected from the group consisting of OH and OAcyl.

11. The antimicrobial agent according to claim 9, in which the compound or one of the compounds of the Formula 1 is selected from the group consisting of:

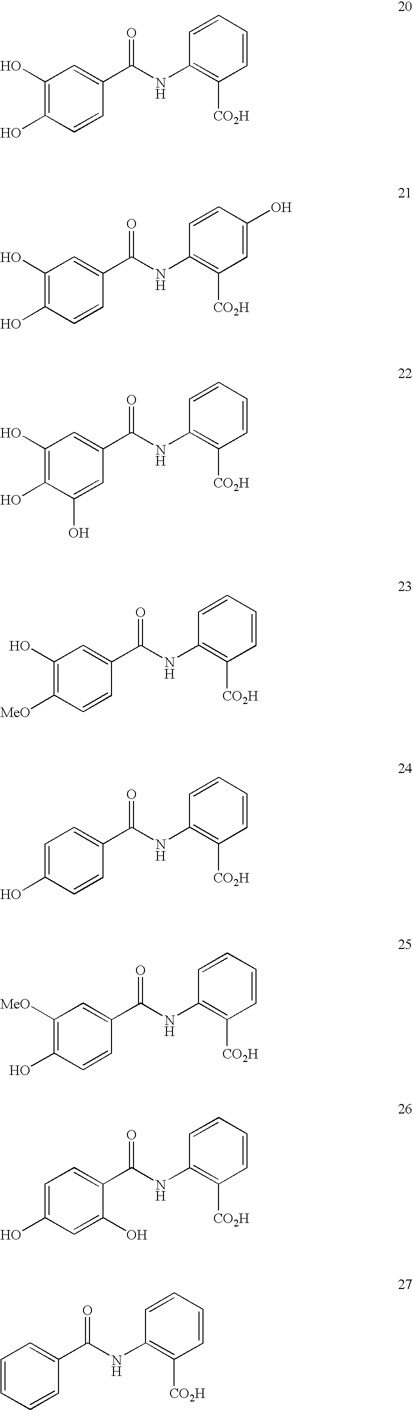

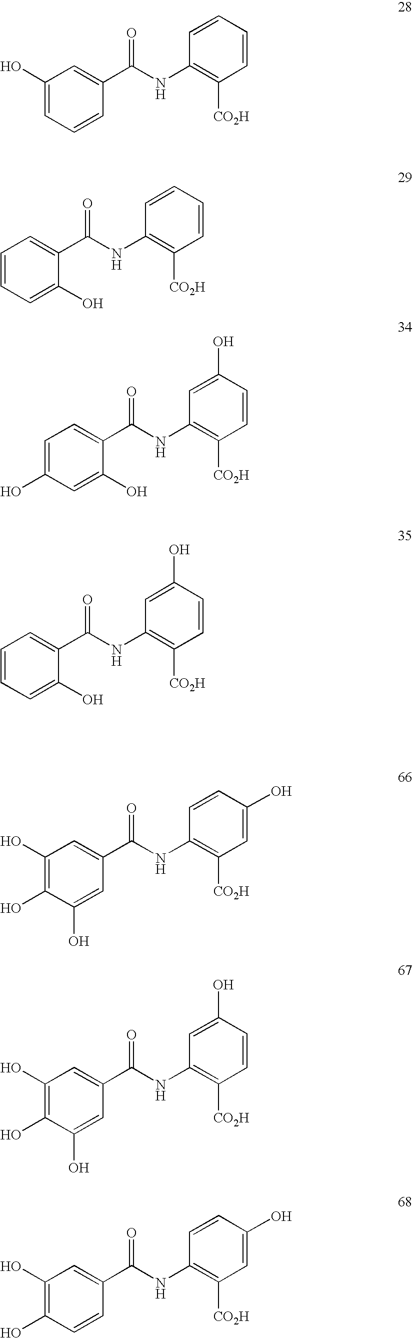

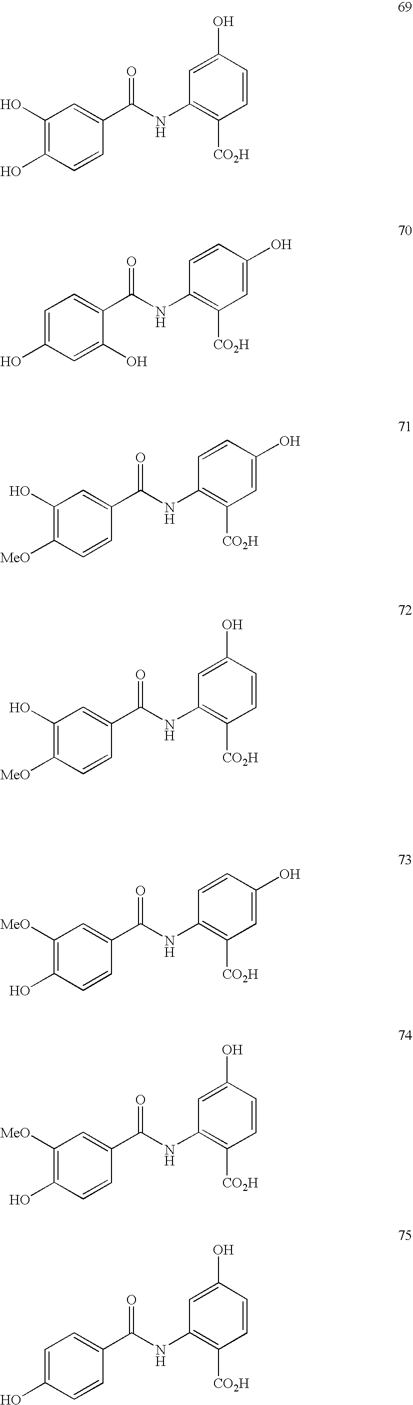

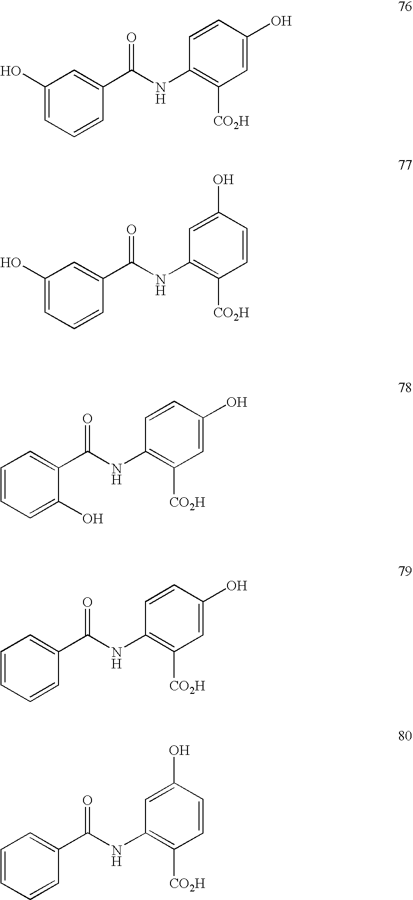

12. The antimicrobial agent according to claim 9, in which for the compounds or one of the compounds of the Formula 1:
n=0,
m=1,
p≦0,
X═OH and
E=H

13. The antimicrobial agent according to claim 12, in which the compound or one of the compounds of the Formula 100 is:

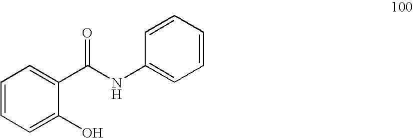

14. The antimicrobial agent according to claim 1, in which:
R³═CH₃ or linear or branched alkyl with 2 to 30 C atoms.

15. Method for inhibiting and/or preventing the growth and/or for destroying microorganisms causing bad breath, comprising the following step:
contacting microorganisms responsible for bad breath with an amount, antimicrobially effective against these microorganisms, of a compound of the Formula 1 or a mixture of two or more compounds of the Formula 1 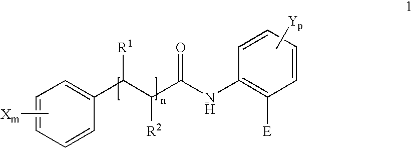
in which for the compound of the Formula 1 or for each compound of the Formula 1 in the mixture:
m=0, 1, 2 or 3,
p=0, 1 or 2,
n=0, 1 or 2,
in which if n=1 or 2 then in each case the pair R¹ and R² in each case denote H or together form a further chemical bond,
in which if m=1, 2 or 3 each X, independently of the other, denotes OH, Oalkyl or Oacyl,
in which if p=1 or 2 each Y, independently of the other, denotes OH, Oalkyl or Oacyl,
in which E=H or denotes a radical —COOR³,
R³═H or alkyl, in which R³═H also for the corresponding pharmaceutically acceptable salts and solvates.

16. Method for controlling and/or preventing bad breath, comprising the following step:
introducing an amount that is antimicrobially effective against microorganisms causing bad breath, of a compound of the Formula 1 or a mixture comprising two or more compounds of the Formula 1 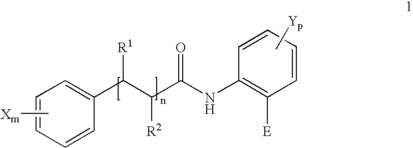
into the oral cavity and/or the pharynx, wherein for the compound of the Formula 1 or each compound of the Formula 1 in the mixture:
m=0, 1, 2, or 3,
p=0, 1 or 2,
n=0, 1 or 2,
in which if n=0 or 2 then in each case the pair R¹ and R² in each case denote H or together form a further chemical bond;
in which if m=1, 2 or 3 each X, independently of the other, denotes OH, Oalkyl or Oacyl,
in which if p=1 or 2 each Y, independently of the other, denotes OH, Oalkyl or Oacyl,
in which E=H or denotes a radical —COOR³,
R³═H or alkyl, in which R³═H also for the corresponding pharmaceutically acceptable salts and solvates.

17. A product that is intended to be introduced into the human oral cavity, to remain there for a specific time and then either to be swallowed or to be removed from the oral cavity, in which the product comprises a compound of the Formula 1 or a mixture of two or more compounds of the Formula 1 according to claim 1 in a sufficient amount to control and/or prevent bad breath.

18. The product according to claim 17, in which the product is a foodstuff or a chewing gum.

19. An oral hygiene product comprising a compound of the Formula 1 or a mixture comprising two or more compounds of the Formula 1 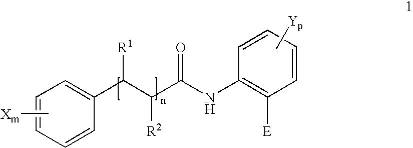
in which for the compound of the Formula 1 or for each compound of the Formula 1 in the mixture,
m=0, 1, 2 or 3,
p=0, 1 or 2,
n=0, 1 or 2,
in which if n=1 or 2 then in each case the pair R¹ and R² in each case denote H or together form a further chemical bond,
in which if m=1, 2 or 3 each X, independently of the other, denotes OH, Oalkyl or Oacyl,
in which if p=1 or 2 each Y, independently of the other, denotes OH, Oalkyl or Oacyl,
in which E=H or denotes a radical —COOR³,
R³═H or alkyl, in which R³═H also for the corresponding pharmaceutically acceptable salts and solvates,
in an amount sufficient to control and/or prevent bad breath.

20. The oral hygiene product according to claim 19, where the product consists essentially of a compound of Formula 1.

21. The oral hygiene product according to claim 19, in which the product is selected from the group consisting of dental cremes, toothpastes, tooth gels, mouthwashes, mouth rinses, liquids for gargling, mouth or throat sprays (pump-action or aerosol sprays), sucking lozenges, sucking tablets, sweets, chewing gums, chewing sweets and dental care chewing gums.

22. A mouthwash containing a mixture comprising:
(a) one or more compounds of the Formula 1A, 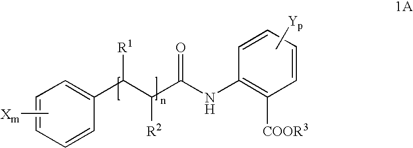
in which for the compound or each compound of the Formula 1A:
m=0, 1, 2 or 3,
p=0, 1 or 2,
n=0, 1 or 2,
in which if n=1 or 2 then in each case the pair R¹ and R² in each case denote H or together form a further chemical bond;
in which if m=1, 2 or 3 each X, independently of the other, denotes OH, Oalkyl or Oacyl,
in which if p=1 or 2 each Y, independently of the other, denotes OH, Oalkyl or Oacyl,
R³═H or alkyl, in which R³═H also for the corresponding pharmaceutically acceptable salts and solvates,
and
(b) one or more cooling substances.

23. The mouthwash according to claim 22, wherein the mixture consists essentially of one or more compounds of Formula 1.
